(19) United States
(12) Patent Application Publication
(10) Pub. No.: US 2008/0200468 A1
(43) Pub. Date: Aug. 21, 2008

(54) 2-Substituted 5-Membered Heteroaryl Carboxylates as Hm74a Receptor Agonists

(75) Inventors: Juliet Kay Simpson, Hertfordshire (GB); Ivan Leo Pinto, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SmithKline Beecham Corporation

(21) Appl. No.: 11/815,389

(22) PCT Filed: Feb. 14, 2006
(86) PCT No.: PCT/GB06/00510

§ 371 Date: Aug. 2, 2007

(30) Foreign Application Priority Data
Feb. 14, 2005 (GB) 0503053.1

(51) Int. Cl. A61K 31/53; C07D 333/36; C07D 401/14; A61P 3/06; A61P 3/00; A61K 31/381

(52) U.S. Cl. 514/243; 549/69; 514/447; 544/183

(57) ABSTRACT

Therapeutically active heteroaryl carboxylic acid derivatives of Formula (I) wherein R¹, R², W, X, Y and Z are as defined in the specification, processes for the preparation of said derivatives, pharmaceutical formulations containing the active compounds and the use of the compounds in therapy, particularly in the treatment of diseases in which under-activation of the HM74A receptor contributes to the disease or in which activation of the receptor will be beneficial, are disclosed.

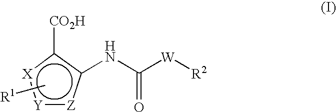

The present invention relates to therapeutically active compounds which are heteroaryl carboxylic acid derivatives, processes for the manufacture of said derivatives, pharmaceutical formulations containing the active compounds and the use of the compounds in therapy, particularly in the treatment of diseases in which under-activation of the HM74A receptor contributes to the disease or in which activation of the receptor will be beneficial.

Dyslipidaemia is a general term used to describe individuals with aberrant lipoprotein profiles. Clinically, the main classes of compounds used for the treatment of patients with dyslipidaemia, and therefore at risk of cardiovascular disease are the statins, fibrates, bile-acid binding resins and nicotinic acid. Nicotinic acid (Niacin, a B vitamin) has been used clinically for over 40 years in patients with various forms of dyslipidaemia. The primary mode of action of nicotinic acid is via inhibition of hormone-sensitive triglyceride lipase (HSL), which results in a lowering of plasma non-esterified fatty acids (NEFA) which in turn alters hepatic fat metabolism to reduce the output of LDL and VLDL (low and very low density lipoprotein). Reduced VLDL levels are thought to lower cholesterol ester transfer protein (CETP) activity to result in increased HDL (high density lipoprotein) levels which may be the cause of the observed cardiovascular benefits. Thus, nicotinic acid produces a very desirable alteration in lipoprotein profiles; reducing levels of VLDL and LDL whilst increasing HDL. Nicotinic acid has also been demonstrated to have disease modifying benefits, reducing the progression and increasing the regression of atherosclerotic lesions and reducing the number of cardiovascular events in several trials.

The observed inhibition of HSL by nicotinic acid treatment is mediated by a decrease in cellular cyclic adenosine monophosphate (cAMP) caused by the G-protein-mediated inhibition of adenylyl cyclase. Recently, the G-protein coupled receptors HM74 and HM74A have been identified as receptors for nicotinic acid (PCT patent application WO02/84298; Wise et. al. J Biol Chem. 2003 278 (11) 9869-9874). The DNA sequence of human HM74A may be found in Genbank; accession number AY148884. Two other papers support this discovery, (Tunaru et. al. Nature Medicine 2003 (3) 352-255 and Soga et. al. Biochem Biophys Res Commun. 2003 303 (1) 364-369), however the nomenclature differs slightly. In the Tunaru paper what they term human HM74 is in fact HM74A and in the Soga paper HM74b is identical to HM74A. Cells transfected to express HM74A and/or HM74 gain the ability to elicit Gᵢ G-protein mediated responses following exposure to nicotinic acid. In mice lacking the homologue of HM74A (m-PUMA-G) nicotinic acid fails to reduce plasma NEFA levels.

We now present a group of substituted 5-membered heteroaryl carboxylic acid derivatives which are selective agonists of the nicotinic acid receptor HM74A and are thus of benefit in the treatment, prophylaxis and suppression of diseases where under-activation of this receptor either contributes to the disease or where activation of the receptor will be beneficial.

### SUMMARY OF THE INVENTION

The present invention provides therapeutically active 5-membered heteroaryl carboxylic acid derivatives, more particularly substituted thiophenecarboxylic acid amide and furancarboxylic acid amide derivatives and the use of these derivatives in therapy, particularly in the treatment of diseases in which under-activation of the HM74A receptor contributes to the disease or in which activation of the receptor will be beneficial, in particular diseases of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity. The compounds may also be of use in the treatment of inflammatory diseases or conditions, as set out further below.

Intermediates, formulations, methods and processes described herein form further aspects of the invention.

### DETAILED DESCRIPTION OF THE INVENTION

The present invention provides a compound of Formula (I)

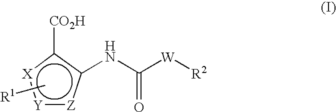

and salts, solvates and physiologically functional derivatives thereof, wherein:

R¹ represents hydrogen or C₁-C₃alkyl;

R² represents a 5, 6 or 10-member aryl, heteroaryl, heterocyclic or alicyclic ring system;

X, Y and Z independently represent S, O or CH, with the proviso that all three of X, Y and Z cannot represent CH;

W represents —(CH₂)_{q}—; —CH═CH—; —(CH₂)ₚNHC(O)—; —(CH₂)ₚNHC(O)NH—; —(CH₂)ₚNHC(O)O—; —(CH₂)ₚSO₂NR³—; —(CH₂)ₚNR³SO₂—; —(CH₂)ₙO—; —C(R⁴R⁵)O— or -A-B-C-;

A and C, which may independently be present or absent, where present independently represent —(CH₂)_{q}—; —CH═CH—; —(CH₂)ₚNHC(O)—; —(CH₂)ₚNHC(O)O—; —(CH₂)ₚNHC(O)NH—; —(CH₂)ₚSO₂NR³—; —(CH₂)ₚNR³SO₂—; —(CH₂)ₚC(O)—; —(CH₂)ₚNH—; —(CH₂)ₚO—; —(CH₂)ₚS— or —(CH₂)ₚO—CH₂—;

B represents a 5 or 6-member aryl, heteroaryl, heterocyclic or alicyclic ring;

n represents an integer selected from 2, 3 and 4;

p represents an integer selected from 0, 1 and 2;

q represents an integer selected from 1, 2, 3 and 4;

R³ represents hydrogen or methyl; and

R⁴ and R⁵, which may be the same or different, independently represent C₁-C₃alkyl.

In certain embodiments of the present invention, R¹ represents hydrogen or methyl.

In certain embodiments, only one of X, Y and Z is a heteroatom, for example, in certain embodiments X, Y and Z, together with the carbon atoms to which they are attached, form a thiophenyl or furanyl ring.

In certain embodiments of the present invention, W represents -A-B-C-, —(CH₂)_{q}—, —(CH₂)ₙO— or —(CH₂)ₚNHC(O)—.

In certain embodiments A represents —O—, —CH₂— or —CH₂O—. In particular embodiments, C is absent or represents —(CH₂)ₚSO₂NR³—, —(CH₂)ₚNHC(O)— or —(CH₂)ₚNHC(O)NH—. In certain embodiments in which A represents —CH₂—, C represents —(CH₂)ₚSO₂NR³—. In certain embodiments in which A represents —O— or —CH₂O—, C is absent.

Particular B groups are 5 or 6 member aryl or heteroaryl rings. In certain embodiments in which B is aryl, for example C6 aryl (e.g. phenyl), B is linked through the 1 and 4 or the 1 and 3 positions. In certain embodiments in which B is heteroaryl, for example a 5 member heteroaryl ring (e.g. 1, 2, 4 oxadiazolyl), B may be linked through the 3 and 5 positions. In other embodiments in which B is heteroaryl, for example a 6 member heteroaryl ring (e.g. pyridinyl), B may be linked through the 2 and 5 positions. When C is —(CH₂)ₚSO₂NR³—, p is 0 and B is unsubstituted phenyl, B may for example be linked through the 1 and 4 (para) positions.

Particular B groups are 5 or 6 member heterocyclic rings. In certain embodiments in which B is C6 heterocyclic (e.g. piperazinyl, piperidinyl), B is linked through the 1 and 4 positions.

In certain embodiments, n represents 2.

In certain embodiments, p represents an integer selected from 0 or 1.

In compounds of the present invention, the R² ring system may be joined to the W linker unit via either a ring carbon atom or via a ring heteroatom, where present.

In certain embodiments in which R² is heteroaryl, R² is selected from pyridinyl, pyrimidinyl, pyridazinyl, pyrazinyl, pyrazolyl, imidazolyl, oxazolyl, isoxazolyl, quinolinyl, cinnolinyl, quinazolinyl and benzotriazinyl. In certain embodiments in which R² is heterocyclic, R² is selected from pyrrolidinyl, imidazolidinyl, piperidinyl and morpholinyl.

In further embodiments, R² is selected from cyclohexyl, phenyl, pyridinyl, pyrimidinyl, pyridazinyl benzotriazinyl and isoxazolyl. As defined below, the 5, 6 or 10-member aryl, heteroaryl, heterocyclic or alicyclic R²groups may be substituted and thus include substituted cyclohexyl, substituted phenyl, substituted pyridine, substituted pyrimidine, substituted pyridazine, substituted benzotriazinyl or substituted isoxazole, in which the substituents are as defined further below.

Thus, where R² is substituted phenyl, the substituents will be those defined for “aryl” substituents below. In some embodiments the substituted phenyl bears one or two substituents selected from halogen C₁₋₃alkyl (for example methylphenyl), C₁₋₃haloalkyl (for example trifluoroalkyl including trifluoromethylphenyl), C₁₋₃alkoxy (for example methoxyphenyl) and C₁₋₃haloakloxy (for example trifluoroalkoxy including trifluoromethoxyphenyl). In certain embodiments in which R² represents singly substituted phenyl, the substituent is at the meta or para position, for example para. In certain embodiments in which R² represents doubly substituted phenyl, the substituents are at the para and meta, or at both meta positions.

In certain embodiments B and R² each represent unsubstituted phenyl, whilst in other embodiments B represents unsubstituted phenyl and R² represents substituted phenyl.

In certain embodiments, R² is selected from the group consisting of:

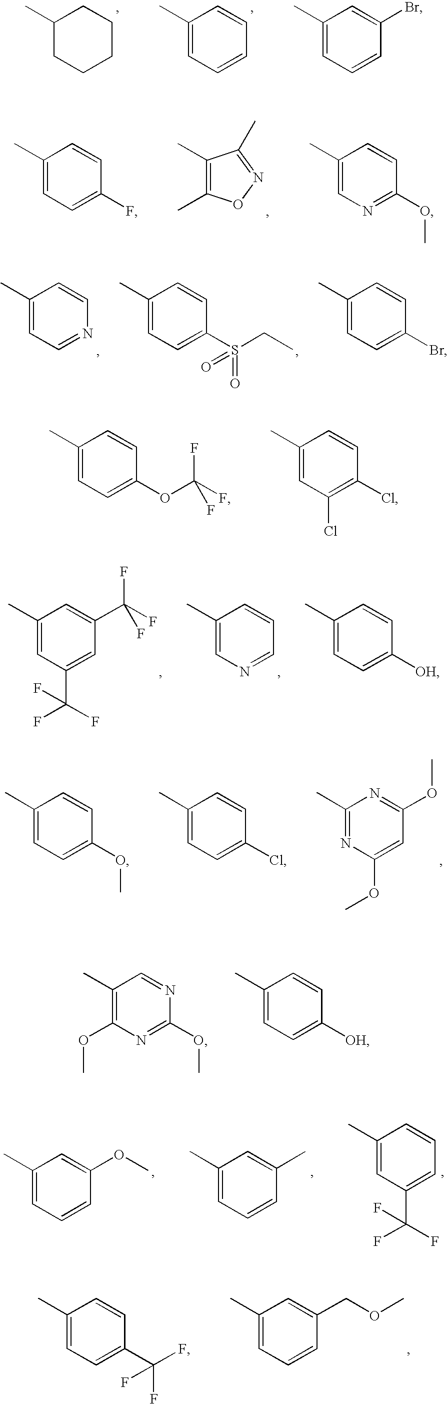

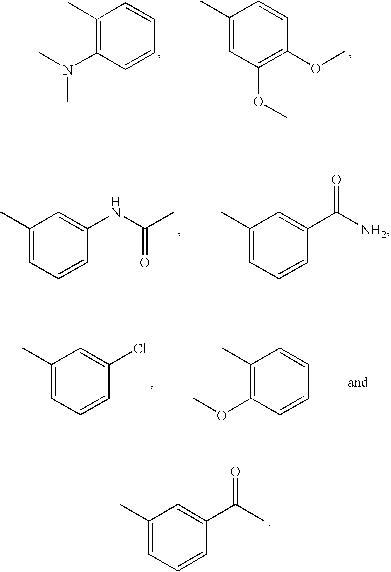

It is to be understood that the present invention includes any combination of particular embodiments and covers all combinations of particular substituents described hereinabove.

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows

As used herein, the terms “halogen” or “halo” refer to fluorine, chlorine, bromine and iodine.

As used herein, the term “alkyl” (when used as a group or as part of a group) refers to an optionally substituted straight or branched hydrocarbon chain containing the specified number of carbon atoms. For example, C₁-C₃alkyl means a straight or branched hydrocarbon chain containing at least 1 and at most 3 carbon atoms. Examples of alkyl as used herein include, but are not limited to; methyl (Me), ethyl (Et), n-propyl, i-propyl and the like. Unless otherwise stated, optional substituents include hydroxy, halogen, ═S and ═O.

As used herein, the term “alkoxy” (when used as a group or as part of a group) refers to an alkyl ether radical, wherein the term “alkyl” is defined above. Examples of alkoxy as used herein include, but are not limited to; methoxy, ethoxy, n-propoxy, i-propoxy and the like.

As used herein, the term “alicyclic” (when used as a group or as part of a group) refers to a cyclic hydrocarbon ring containing the specified number of carbon atoms. Examples of alicyclic as used herein include, but are not limited to cyclohexyl, cyclopropyl and the like. Said alicyclic groups may be optionally substituted with one or more, for example 1 to 3, groups selected from hydroxy, halogen, ═S, ═O, C₁-C₃alkyl (which may be further substituted with one or more hydroxy, ═O or halo groups), optionally halogenated C₁-C₃alkoxy, C₁-C₃alkoxyC₁-C₃alkyl, NR³₂, —NHC(O)C₁-C₃alkyl, —C(O)NR³₂, and —S(O)₂C₁-C₃alkyl, wherein R³ is as defined above.

As used herein, the term “aryl” (when used as a group or as part of a group) refers to an aromatic hydrocarbon ring of the specified number of carbons. Examples of aryl as used herein include, but are not limited to, phenyl, naphthyl and benzyl. Said aryl groups may be optionally substituted with one or more, for example 1 to 3 groups selected from hydroxy, halogen, ═S, ═O, C₁-C₃alkyl (which may be further substituted with one or more hydroxy, ═O or halo groups), optionally halogenated C₁-C₃alkoxy, C₁-C₃alkoxyC₁-C₃alkyl, NR³₂, —NHC(O)C₁-C₃alkyl, —C(O)NR³₂, and —S(O)₂C₁-C₃alkyl, wherein R³ is as defined above.

As used herein, the term “heteroaryl” (when used as a group or as part of a group) refers to an aryl group, as defined above, which contains one or more sulphur, nitrogen or oxygen heteroatoms. Examples of heteroaryl as used herein include, but are not limited to, thiophene, furan, pyridine, pyrimidine, pyridazine, imidazole, isoxazole, oxadiazoles, quinolines, benzotriazines and the like. Said heteroaryl groups may be optionally substituted with one or more, for example 1 to 3 groups selected from hydroxy, halogen, ═S, ═O, C₁-C₃alkyl (which may be further substituted with one or more hydroxy, ═O or halo groups), optionally halogenated C₁-C₃alkoxy, C₁-C₃alkoxyC₁-C₃alkyl, NR³₂, —NHC(O)C₁-C₃alkyl, —C(O)NR³₂, and —S(O)₂C₁-C₃alkyl, wherein R³ is as defined above.

As used herein, the term “heterocyclic” (when used as a group or as part of a group) refers to an alicyclic group, as defined above, which contains one or more nitrogen or oxygen heteroatoms. Said heterocyclic groups may be optionally substituted with one or more, for example 1 to 3 groups selected from hydroxy, halogen, ═S, ═O, C₁-C₃alkyl (which may be further substituted with one or more hydroxy, ═O or halo groups), optionally halogenated C₁-C₃alkoxy, C₁-C₃alkoxyC₁-C₃alkyl, NR³₂, —NHC(O)C₁-C₃alkyl, —C(O)NR³₂, and —S(O)₂C₁-C₃alkyl, wherein R³ is as defined above.

As used herein, the term “physiologically functional derivative” refers to any pharmaceutically acceptable derivative of a compound of the present invention, for example an ester or an amide thereof, and includes any pharmaceutically acceptable salt, ester, or salt of such ester of a compound of Formula (I) which, upon administration to a mammal, such as a human, is capable of providing (directly or indirectly) a compound of Formula (I) or an active metabolite or residue thereof. It will be appreciated by those skilled in the art that the compounds of Formula (I) may be modified to provide physiologically functional derivatives thereof at any of the functional groups in the compounds, and that the compounds of Formula (I) may be so modified at more than one position.

As used herein, the term “pharmaceutically acceptable” used in relation to an ingredient (active ingredient or excipient) which may be included in a pharmaceutical formulation for administration to a patient, refers to that ingredient being acceptable in the sense of being compatible with any other ingredients present in the pharmaceutical formulation and not being deleterious to the recipient thereof.

As used herein, the term “solvate” refers to a complex of variable stoichiometry formed by a solute (in this invention, a compound of Formula (I), a salt thereof or a physiologically functional derivative thereof) and a solvent. Such solvents for the purposes of the present invention may not interfere with the biological activity of the solute. Examples of suitable solvents include water, methanol, ethanol and acetic acid. Preferably the solvent used is a pharmaceutically acceptable solvent. Examples of suitable pharmaceutically acceptable solvents include water, ethanol and acetic acid. Most preferably the solvent used is water, in which case the solvate may be referred to as a hydrate of the solute in question.

It will be appreciated that, for pharmaceutical use, the “salt or solvate” referred to above will be a pharmaceutically acceptable salt or solvate. However, other salts or solvates may find use, for example, in the preparation of a compound of Formula (I) or in the preparation of a pharmaceutically acceptable salt or solvate thereof.

Pharmaceutically acceptable salts include those described by Berge, Bighley and Monkhouse, J. Pharm. Sci., 1977, 66, 1-19. Suitable pharmaceutically acceptable salts include acid addition salts formed from the addition of inorganic acids or organic acids, preferably inorganic acids. Examples of suitable acid addition salts include hydrochlorides, hydrobromides, sulphates and acetates. Further representative examples of pharmaceutically acceptable salts include those formed from maleic, fumaric, benzoic, ascorbic, pamoic, succinic, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids. Suitable pharmaceutically acceptable salts also include alkali metal salts formed from the addition of alkali metal bases such as alkali metal hydroxides. An example of a suitable alkali metal salt is a sodium salt.

In a further aspect, the present invention provides the use of compounds of Formula (I)

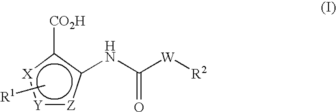

or a salt, solvate and physiologically functional derivative thereof as defined above in the manufacture of a medicament for the treatment of disorders of lipid metabolism, including dislipidaemia or hyperlipoproteinaemia, or of inflammatory diseases or conditions. It is to be understood that this aspect of the present invention includes, with respect to the use of compounds of Formula (I) in the manufacture of a medicament, any combination of particular embodiments and covers all combinations of particular substituents of compounds of Formula (I) described hereinabove.

Compounds of the present invention are of potential therapeutic benefit in the treatment and amelioration of the symptoms of many diseases of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity. The use of a compound of Formula (I) in the treatment of one or more of these diseases is a further aspect of the present invention.

Furthermore, it is also believed that the HM74 and HM74A receptors are involved in inflammation. Inflammation represents a group of vascular, cellular and neurological responses to trauma. Inflammation can be characterised as the movement of inflammatory cells such as monocytes, neutrophils and granulocytes into the tissues. This is usually associated with reduced endothelial barrier function and oedema into the tissues. Inflammation with regards to disease typically is referred to as chronic inflammation and can last up to a lifetime. Such chronic inflammation may manifest itself through disease symptoms. The aim of anti-inflammatory therapy is therefore to reduce this chronic inflammation and allow for the physiological process of healing and tissue repair to progress.

Thus, a further aspect of the present invention resides in the use of a compound of Formula (I) or a salt, solvate or physiologically functional derivative thereof as defined above in the treatment of inflammatory diseases or conditions of the joint, particularly arthritis (e.g. rheumatoid arthritis, osteoarthritis, prosthetic joint failure), or the gastrointestinal tract (e.g. ulcerative colitis, Crohn's disease, and other inflammatory bowel and gastrointestinal diseases, gastritis and mucosal inflammation resulting from infection, the enteropathy provoked by non-steroidal anti-inflammatory drugs), of the lung (e.g. adult respiratory distress syndrome, asthma, cystic fibrosis, or chronic obstructive pulmonary disease), of the heart (e.g. myocarditis), of nervous tissue (e.g. multiple sclerosis), of the pancreas, (e.g. inflammation associated with diabetes melitus and complications thereof), of the kidney (e.g. glomerulonephritis), of the skin (e.g. dermatitis, psoriasis, eczema, urticaria, burn injury), of the eye (e.g. glaucoma) as well as of transplanted organs (e.g. rejection) and multi-organ diseases (e.g. systemic lupus erythematosis, sepsis) and inflammatory sequelae of viral or bacterial infections and inflammatory conditions associated with atherosclerosis and following hypoxic or ischaemic insults (with or without reperfusion), for example in the brain or in ischaemic heart disease.

In particular, the compounds of Formula (I) are useful in the treatment and prevention of inflammation, and cardiovascular diseases or conditions including atherosclerosis, arteriosclerosis, hypertriglyceridemia, and mixed dyslipidaemia.

Thus, there is also provided the use of a compound of Formula (I) or a pharmaceutically acceptable salt, solvate or physiologically functional derivative thereof, in the manufacture of a medicament for the treatment of disorders of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia. The compounds are also provided for use in the treatment of coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity.

Nicotinic acid has a significant side effect profile, possibly because it is dosed at high level (gram quantities daily). The most common side effect is an intense cutaneous flushing. The compounds of the present invention preferably exhibit reduced side effects compared to nicotinic acid.

HM74A has been identified as a high affinity receptor for nicotinic acid whilst HM74 is a lower affinity receptor. Desirably, the compounds of the present invention are selective for HM74A by which is meant that they show greater affinity for HM74A than for HM74.

The potential for compounds of Formula (I) to activate HM74A may be demonstrated, for example, using the following in vitro and in vivo assays:

In-Vitro Testing

For transient transfections, HEK293T cells (HEK293 cells stably expressing the SV40 large T-antigen) are maintained in DMEM containing 10% foetal calf serum and 2mM glutamine. Cells are seeded in 90 mm culture dishes and grown to 60-80% confluence (18-24 h) prior to transfection. Human HM74A (GenBank™ accession number AY148884) is subcloned in to a mammalian expression vector (pcDNA3; Invitrogen) and transfected using Lipofectamine reagent. For transfection, 9 μg of DNA is mixed with 30 μl Lipofectamine in 0.6 ml of Opti-MEM (Life Technologies Inc.) and incubated at room temperature for 30 min prior to the addition of 1.6 ml of Opti-MEM. Cells are exposed to the Lipofectamine/DNA mixture for 5 h and 6 ml of 20% (v/v) foetal calf serum in DMEM is then added. Cells are harvested 48 h after transfection. Pertussis toxin treatment is carried out by supplementation into media at 50 ngml⁻¹ for 16 h. All transient transfection studies involve co-transfection of receptor together with the G_{i/o} G protein, G₀₁α.

For generation of stable cell lines the above method is used to transfect CHO-K1 cells seeded in six well dishes grown to 30% confluence. These cells are maintained in DMEM F-12 HAM media containing 10% foetal calf serum and 2 mM glutamine. 48 h post-transfection the media is supplemented with 400 μg/ml Geneticin (G418, Gibco) for selection of antibiotic resistant cells. Clonal CHO-K1 cell lines stably expressing HM74A are confirmed by [³⁵S]-GTPγS binding measurements, following the addition of nicotinic acid.

P2 membrane preparation—Plasma membrane-containing P2 particulate fractions are prepared from cell pastes frozen at −80° C. after harvest. All procedures are carried out at 4° C. Cell pellets are resuspended in 1 ml of 10 mM Tris-HCl and 0.1 mM EDTA, pH 7.5 (buffer A) and by homogenisation for 20 s with a Ultra Turrax followed by passage (5 times) through a 25-gauge needle. Cell lysates are centrifuged at 1,000 g for 10 min in a microcentrifuge to pellet the nuclei and unbroken cells and P2 particulate fractions are recovered by microcentrifugation at 16,000 g for 30 min. P2 particulate fractions are resuspended in buffer A and stored at −80° C. until required.

[³⁵S]-GTPγS binding—Assays are performed at room temperature either in 96-well format as described previously (Wieland, T. and Jakobs, K. H. (1994) Methods Enzymol. 237, 3-13) or in an adapted protocol carried out in 384-well format.

96-well format: Briefly, membranes (10 μg per point) are diluted to 0.083 mg/ml in assay buffer (20 mM HEPES, 100 mM NaCl, 10 mM MgCl₂, pH7.4) supplemented with saponin (10 mg/l) and pre-incubated with 10 μM GDP. Various concentrations of nicotinic acid or related molecules are added, followed by [³⁵S]-GTPγS (1170 Ci/mmol, Amersham) at 0.3 nM (total vol. of 100 μl) and binding is allowed to proceed at room temperature for 30 min. Non-specific binding is determined by the inclusion of 0.6 mM GTP. Wheatgerm agglutinin SPA beads (Amersham) (0.5 mg) in 25 μl assay buffer are added and the whole is incubated at room temperature for 30 min with agitation. Plates are centrifuged at 1500 g for 5 min and bound [³⁵S]-GTPγS is determined by scintillation counting on a Wallac 1450 microbeta Trilux scintillation counter.

384-well format: Briefly, the dilution of standard or test compounds are prepared and added to a 384-well plate in a volume of 10 μl. Membranes (HM74A or HM74) are diluted in assay buffer (20 mM HEPES, 100 mM NaCl, 10 mM MgCl₂, pH7.4) supplemented with saponin (60 μg/ml), Leadseeker WGA beads (Amersham; 250 μg/well) and 10 μM GDP, so that the 20 μl volume added to each well contains 5 μg of membranes. [³⁵S]-GTPzγS (1170 Ci/mmol, Amersham) is diluted (1:1500) in assay buffer and 20 μl added to each well. Following the addition of the radioligand, the plates are sealed, pulse spun and incubated for 4 hours at room temperature. At the end of the incubation period the plates are read on a Leadseeker machine (VIEWLUX PLUS; Perkin-Elmer) to determine the levels of specific binding.

In-Vivo Testing

HM74A agonists are tested in male Spague-Dawley rats (200-250 grammes) which have been fasted for at least 12 hours prior to the study. The compounds are dosed intravenously (5 ml/kg) or by oral gavage (10 ml/kg). Blood samples (0.3 ml tail vein bleed) are taken pre-dose and at three times post-dose (times ranging from 15 minutes to 8 hours post-dose). Each blood sample is transferred to a heparin tube (Becton Dickinson Microtainer, PST LH) and centrifuged (10,000 g for 5 minutes) to produce a plasma sample. The plasma samples are assayed for levels of non-esterified fatty acids (NEFA) using a commercially available kit (Randox). Inhibition of plasma NEFA levels, relative to pre-dose levels, is used as a surrogate for HM74A agonist activity.

In order to determine whether compounds of the invention exhibit the flushing response associated with nicotinic acid, they are dosed to anaesthetised guinea-pigs. Nicotinic acid is used as positive control. Male Dunkin Hartley guinea pigs (300-800 g) are fasted for 12 hours prior to being anaesthetised with a mixture of Ketamine hydrochloride (Vetalar, 40 mg/kg i.m.), Xylazine (Rompun, 8 mg/kg i.m.) and sodium pentobarbitone (Sagatal, 30 mg/kg i.p.). Following anaesthesia a tracheostomy is performed and the animals are mechanically ventilated with room air (10-12 mL/kg, 60 breaths/min). A jugular vein, and a carotid artery, are cannulated for intravenous administration of test compound and collection of blood respectively. An infra-red temperature probe (Extech Instruments) is placed 3-5 mm from the tip of the left ear. Temperature measurements are recorded every minute from 5 minutes prior to test compound or nicotinic acid and up to 40 minutes post-administration of test compound or nicotinic acid. Data is automatically collected on a Psion computer before being transferred for data analysis within an Excel spreadsheet. Prior to, and at frequent time points after compound administration, blood samples (0.3 ml) are taken via the carotid arterial cannula and transferred to Microtainer (BD) tubes containing lithium heparin. The samples are mixed thoroughly on a blood roller and then stored on ice prior to centrifugation at 1200g for 5 minutes.

As indicated above, compounds of Formula (I) are useful in human or veterinary medicine, in particular as activators of HM74A, in the management of dyslipidaemia and hyperlipoproteinaemia.

Thus, there is provided as a further aspect of the present invention a compound of Formula (I) or a pharmaceutically acceptable salt, solvate or physiologically functional derivative thereof, for use in human or veterinary medicine, particularly in the treatment of disorders of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, and hypertriglyceridaemia. As such, the compounds may also find favour as therapeutics for coronary artery disease, thrombosis, angina, chronic renal failure, peripheral vascular disease and stroke, as well as the cardiovascular indications associated with type II diabetes mellitus, type I diabetes, insulin resistance, hyperlipidaemia, anorexia nervosa, obesity.

It will be appreciated that references herein to treatment extend to prophylaxis, prevention of recurrence and suppression of symptoms as well as the treatment of established conditions.

In a further or alternative aspect there is provided a method for the treatment of a human or animal subject with a condition in which under-activation of the HM74A receptor contributes to the condition or in which activation of the receptor will be beneficial, which method comprises administering to said human or animal subject an effective amount of a compound of Formula (I) or a physiologically acceptable salt or solvate thereof.

More particularly, the present invention provides a method for the treatment of disorders of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, or hypertriglyceridaemia which method comprises administering to said human or animal subject an effective amount of a compound of Formula (I) or a physiologically acceptable salt or solvate thereof. The invention also provides methods for the treatment of coronary artery disease, thrombosis, angina, chronic renal failure, peripheral vascular disease or stroke, as well as the cardiovascular indications associated with type II diabetes mellitus, type I diabetes, insulin resistance, hyperlipidaemia, anorexia nervosa, obesity which methods comprise administering to said human or animal subject an effective amount of a compound of Formula (I) or a physiologically acceptable salt, solvate or derivative thereof.

The amount of a HM74A modulator which is required to achieve the desired biological effect will, of course, depend on a number of factors, for example, the mode of administration and the precise clinical condition of the recipient. In general, the daily dose will be in the range of 0.1 mg-1 g/kg, typically 0.1-100 mg/kg. An intravenous dose may, for example, be in the range of 0.01 mg to 0.1 g/kg, typically 0.01 mg to 10 mg/kg, which may conveniently be administered as an infusion of from 0.1 μg to 1 mg, per minute. Infusion fluids suitable for this purpose may contain, for example, from 0.01 μg to 0.1 mg, per millilitre. Unit doses may contain, for example, from 0.01 μg to 1 g of a HM74A modulator. Thus ampoules for injection may contain, for example, from 0.01 μg to 0.1 g and orally administrable unit dose formulations, such as tablets or capsules, may contain, for example, from 0.1 mg to 1 g. No toxicological effects are indicated/expected when a compound of the invention is administered in the above mentioned dosage range.

A compound of the present invention may be employed as the compound per se in the treatment of a the treatment of diseases where under-activation of the HM74A receptor contributes to the disease or where activation of the receptor will be beneficial, but is preferably presented with an acceptable carrier in the form of a pharmaceutical formulation. The carrier must, of course, be acceptable in the sense of being compatible with the other ingredients of the formulation and must not be deleterious to the recipient. The carrier may be a solid or a liquid, or both, and is preferably formulated with the HM74A modulator as a unit-dose formulation, for example, a tablet, which may contain from 0.05% to 95% by weight of the HM74A modulator.

The formulations include those suitable for oral, rectal, topical, buccal (e.g. sub-lingual) and parenteral (e.g. subcutaneous, intramuscular, intradermal or intravenous) administration.

There is also provided according to the invention a process for preparation of such a pharmaceutical composition which comprises mixing the ingredients.

Formulations suitable for oral administration may be presented in discrete units, such as capsules, cachets, lozenges or tablets, each containing a predetermined amount of a HM74A modulator; as a powder or granules; as a solution or a suspension in an aqueous or non-aqueous liquid; or as an oil-in-water or water-in-oil emulsion. In general, the formulations are prepared by uniformly and intimately admixing the active HM74A modulator with a liquid or finely divided solid carrier, or both, and then, if necessary, shaping the product. For example, a tablet may be prepared by compressing or moulding a powder or granules of the HM74A modulator optionally with one or more accessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Moulded tablets may be made by moulding, in a suitable machine, the powdered compound moistened with an inert liquid diluent.

Tablets and capsules for oral administration may contain conventional excipients such as binding agents, for example syrup, acacia, gelatin, sorbitol, tragacanth, mucilage of starch or polyvinyl pyrrolidone; fillers, for example, lactose, microcrystalline cellulose, sugar, maize- starch, calcium phosphate or sorbitol; lubricants, for example, magnesium stearate, stearic acid, talc, polyethylene glycol or silica; disintegrants, for example, potato starch, croscarmellose sodium or sodium starch glycollate; or wetting agents such as sodium lauryl sulphate. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxymethyl cellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example almond oil, fractionated coconut oil, oily esters, propylene glycol or ethyl alcohol; or preservatives, for example, methyl or propyl p-hydroxybenzoates or sorbic acid. The preparations may also contain buffer salts, flavouring, colouring and/or sweetening agents (e.g. mannitol) as appropriate.

Formulations suitable for buccal (sub-lingual) administration include lozenges comprising a HM74A modulator in a flavoured base, usually sucrose and acacia or tragacanth, and pastilles comprising the HM74A modulator in an inert base such as gelatin and glycerin or sucrose and acacia.

Formulations of the present invention suitable for parenteral administration conveniently comprise sterile aqueous preparations of an HM74A modulator, preferably isotonic with the blood of the intended recipient. These preparations are preferably administered intravenously, although administration may also be effected by means of subcutaneous, intramuscular, or intradermal injection. Such preparations may conveniently be prepared by admixing the HM74A modulator with water and rendering the resulting solution sterile and isotonic with the blood. Injectable compositions according to the invention will generally contain from 0.1 to 5% w/w of the HM74A modulator.

Thus, formulations of the present invention suitable for parenteral administration comprising a compound according to the invention may be formulated for parenteral administration by bolus injection or continuous infusion and may be presented in unit dose form, for instance as ampoules, vials, small volume infusions or pre-filled syringes, or in multi-dose containers with an added preservative. The compositions may take such forms as solutions, suspensions, or emulsions in aqueous or non-aqueous vehicles, and may contain formulatory agents such as anti-oxidants, buffers, antimicrobial agents and/or toxicity adjusting agents. Alternatively, the active ingredient may be in powder form for constitution with a suitable vehicle, e.g. sterile, pyrogen-free water, before use. The dry solid presentation may be prepared by filling a sterile powder aseptically into individual sterile containers or by filling a sterile solution aseptically into each container and freeze-drying.

Formulations suitable for rectal administration are preferably presented as unit-dose suppositories. These may be prepared by admixing a HM74A modulator with one or more conventional solid carriers, for example, cocoa butter or glycerides and then shaping the resulting mixture.

Formulations suitable for topical application to the skin preferably take the form of an ointment, cream, lotion, paste, gel, spray, aerosol, or oil. Carriers which may be used include vaseline, lanolin, polyethylene glycols, alcohols, and combinations of two or more thereof. The HM74A modulator is generally present at a concentration of from 0.1 to 15% w/w of the composition, for example, from 0.5 to 2%.

By topical administration as used herein, we include administration by insufflation and inhalation. Examples of various types of preparation for topical administration include ointments, creams, lotions, powders, pessaries, sprays, aerosols, capsules or cartridges for use in an inhaler or insufflator or drops (e.g. eye or nose drops).

Ointments and creams may, for example, be formulated with an aqueous or oily base with the addition of suitable thickening and/or gelling agents and/or solvents. Such bases may thus, for example, include water and/or an oil such as liquid paraffin or a vegetable oil such as arachis oil or castor oil or a solvent such as a polyethylene glycol. Thickening agents which may be used include soft paraffin, aluminium stearate, cetostearyl alcohol, polyethylene glycols, microcrystalline wax and beeswax.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents or suspending agents.

Spray compositions may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2- tetrafluorethane, carbon dioxide or other suitable gas.

Capsules and cartridges for use in an inhaler or insufflator, of for example gelatin, may be formulated containing a powder mix of a compound of the invention and a suitable powder base such as lactose or starch.

The pharmaceutical compositions according to the invention may also be used in combination with other therapeutic agents, for example in combination with other classes of dyslipidaemic drugs (e.g. statins, fibrates, bile-acid binding resins or nicotinic acid).

The compounds of the instant invention may be used in combination with one or more other therapeutic agents for example in combination with other classes of dyslipidaemic drugs e.g. 3-hydroxy-3-methylglutaryl-coenzyme A reductase inhibitors (statins) or fibrates or bile acid binding resins or nicotinic acid. The invention thus provides, in a further aspect, the use of such a combination in the treatment of diseases in which under-activation of the HM74A receptor contributes to the disease or in which activation of the receptor will be beneficial and the use of a compound of Formula (I) or a pharmaceutically acceptable salt, solvate or physiologically functional derivative thereof in the manufacture of a medicament for the combination therapy of disorders of lipid metabolism including dislipidaemia or hyperlipoproteinaemia such as diabetic dyslipidaemia and mixed dyslipidaemia, heart failure, hypercholesterolaemia, cardiovascular disease including atherosclerosis, arteriosclerosis, or hypertriglyceridaemia, coronary artery disease, thrombosis, angina, chronic renal failure, peripheral vascular disease or stroke, as well as the cardiovascular indications associated with type II diabetes mellitus, type I diabetes, insulin resistance, hyperlipidaemia, anorexia nervosa, obesity.

When the compounds of the present invention are used in combination with other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same formulation it will be appreciated that the two components must be stable and compatible with each other and the other components of the formulation and may be formulated for administration. When formulated separately they may be provided in any convenient formulation, conveniently in such a manner as are known for such compounds in the art.

When in combination with a second therapeutic agent active against the same disease, the dose of each component may differ from that when the compound is used alone. Appropriate doses will be readily appreciated by those skilled in the art.

The invention thus provides, in a further aspect, a combination comprising a compound of Formula (I) or a physiologically acceptable salt or solvate thereof together with another therapeutically active agent. The combination may conveniently be presented for use in the form of a pharmaceutical formulation and thus pharmaceutical formulations comprising a combination as defined above together with a pharmaceutically acceptable carrier thereof represent a further aspect of the invention.

Compounds of Formula (I) and salts and solvates thereof may be prepared by various synthetic routes, including the methodology described hereinafter which constitutes a further aspect of the invention.

Abbreviations
PyHOTs Pyridinium tosylate
CDI Carbonyl diimidazole
THF Tetrahydrofuran
DMF N,N-dimethylformamide
PyHCl Pyridinium hydrochloride
DIPEA N,N-diisopropylethylamine
IPA Isopropyl alcohol

Method A:

A process for preparing compounds of Formula (I)

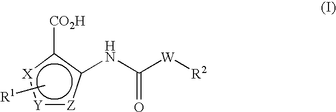

in which R¹ represents C₁₋₃alkyl and W, X, Y, Z and R² are as defined above, is set out in scheme (a):

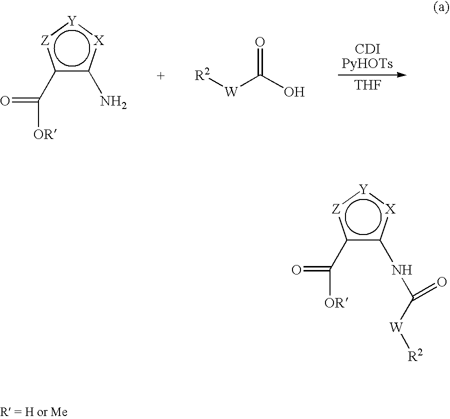

Accordingly, the present invention provides a process for preparing a compound of Formula (I) comprising amide coupling using CDI and, where desired or necessary, converting a resultant free acid or base compound of Formula (I) into a physiologically acceptable salt form, or vice versa, or converting one salt form into another physiologically acceptable salt form.

Method B:

Conversion of a methyl ester of a compound of Formula (I) to the corresponding carboxylic acid may be carried out using pyridine hydrochloride as set out in scheme (b):

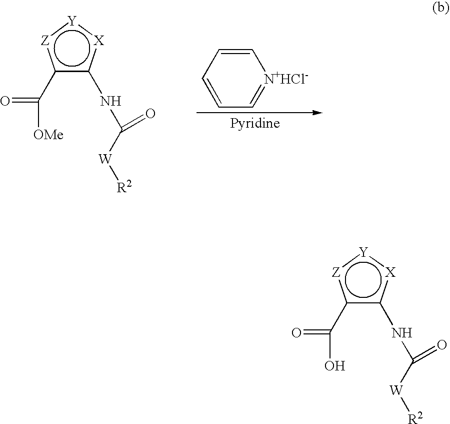

Method C:

A process for preparing compounds of Formula (I)

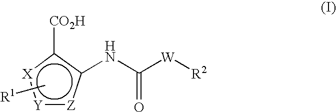

in which R¹ represents hydrogen and W, X, Y, Z and R² are as defined above, is set out in scheme (c):

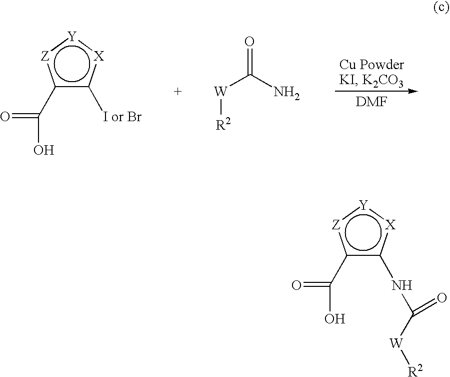

Accordingly, the present invention provides a process for preparing a compound of Formula (I) comprising coupling of the iodo or bromo heterocycle with an amide using copper and, where desired or necessary, converting a resultant free acid or base compound of Formula (I) into a physiologically acceptable salt form, or vice versa, or converting one salt form into another physiologically acceptable salt form.

Method D:

A process for preparing compounds of Formula (I)

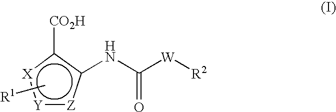

in which R¹ represents hydrogen, X represents S, Y and Z each represent CH, W and R² are as defined above, is set out in scheme (d):

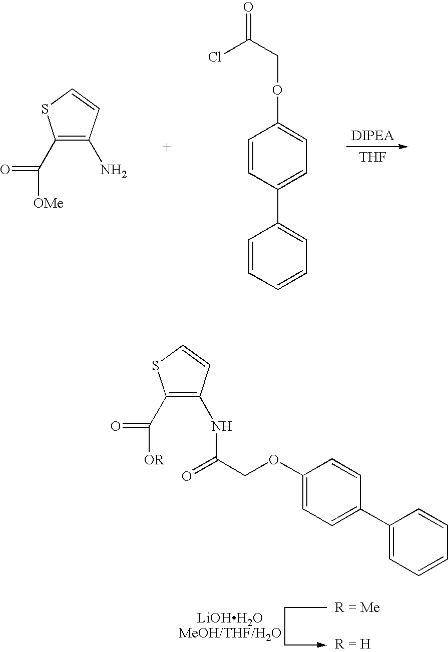

Accordingly, the present invention provides a process for preparing a compound of Formula (I) comprising formation of an amide bond between an amino thiophene and an acid chloride followed by base hydrolysis of the methyl ester.

Method E:

A process for preparing compounds of Formula (I)

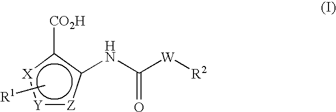

in which R¹ represents hydrogen, Y represents S, X and Z each represent CH, W represents piperazinyl and R² is as defined above, is set out in scheme (e):

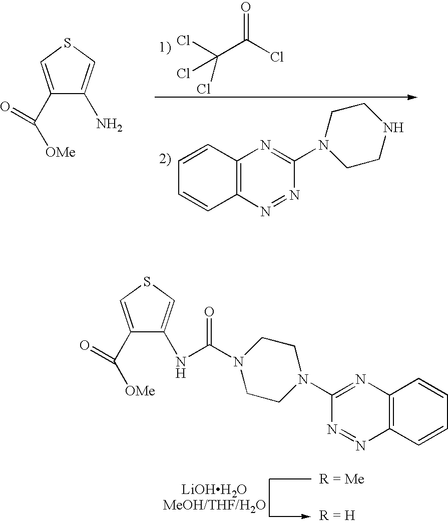

Accordingly, the present invention provides a process for preparing a compound of Formula (I) comprising:
a) Reaction of an amino thiophene with trichloromethylchloroformate then reaction with substituted piperazine.
b) Hydrolysis of the methyl ester using base.

Method F:

A process for preparing compounds of Formula (I)

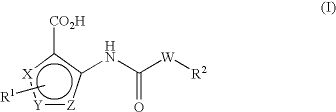

in which R¹ represents hydrogen, X and Y are as defined, Z represents CH, W represents -A-B-C-, A represents —(CH₂)_{q}—, B represents a cyclic amine, C is absent and R² represents benzotriazinyl is set out in scheme (f):

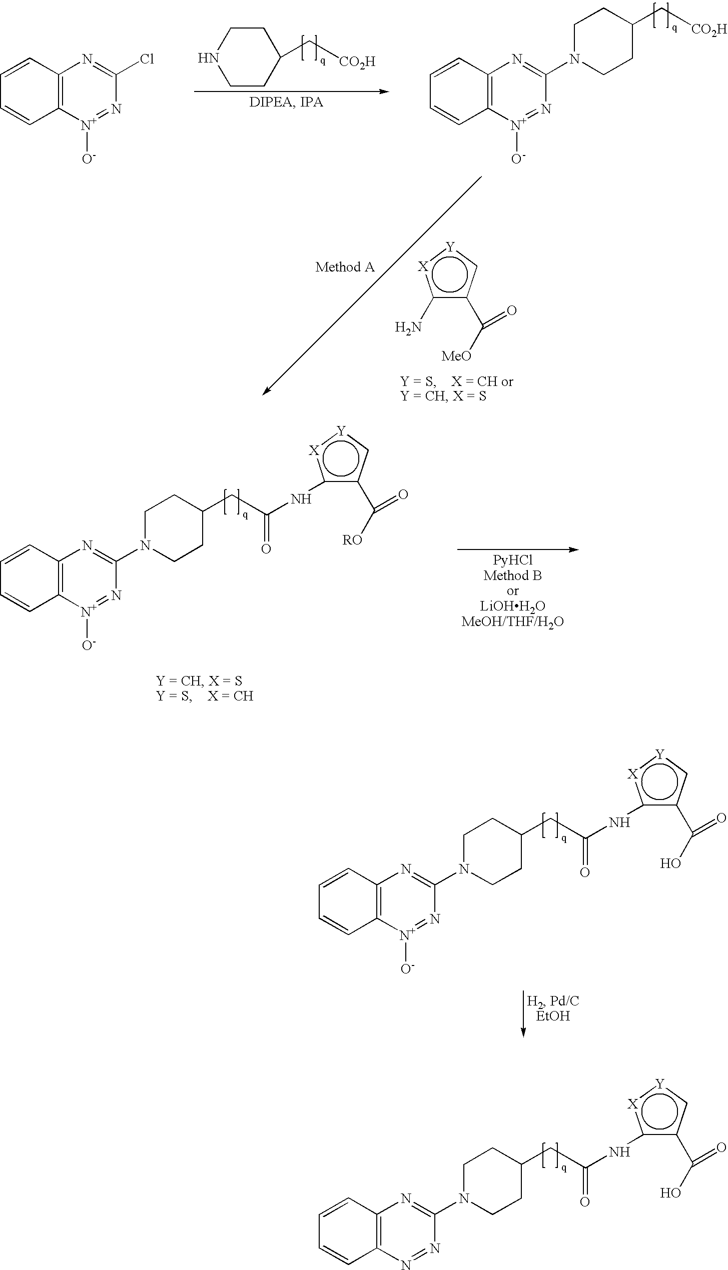

Accordingly, the present invention provides a process for preparing a compound of Formula (I) comprising:
a) Displacement of chloride with an amine.
b) Amide coupling using CDI (Method A)
c) Conversion of methyl ester to carboxylic acid using pyridine hydrochloride (Method B) or base hydrolysis.

The following non-limiting examples illustrate the present invention:

### SYNTHETIC EXAMPLES

### Example 1

### 4-{[(4-Biphenylyloxy)acetyl]amino}-3-thiophenecarboxylic acid

### a) Methyl-4-{[(4-biphenylyloxy)acetyl]amino}-3-thiophenecarboxylate

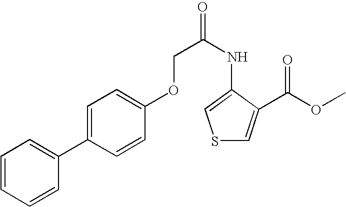

(Method A)

(4-biphenylyloxy)acetic acid (J. Med. Chem 1976, 19, 1079-1088) (0.151 g, 0.663 mmol, 1 equiv) and 1,1-carbonyldiimidazole (0.129 g, 0.795 mmol, 1.2 equiv) were stirred vigorously in anhydrous THF (5 ml) under nitrogen. After 1 hour, methyl-4-amino thiophene-3-carboxylate (0.125 g, 0.795 mmol, 1.2 equiv) and pyridinium p-toluene sulfonate (400 mg, 2.16 mmol, 2.4 equiv) were added and the mixture was refluxed under nitrogen for 18-20 hours. The mixture was then cooled to room temperature, filtered through Celite and concentrated in vacuo to afford the crude product. Purification by Biotage™ chromatography (Si, 50 g) eluting 19-9:1 cyclohexane:ethylacetate afforded the title compound as a white solid (0.052 g, 21%). ¹H NMR δ_{H} (400 MHz; CDCl₃) δ: 3.94 (3H, s), 4.71 (2H, s), 7.15 (2H, m), 7.33 (1H, m), 7.44 (2H, app t, J=7.5 Hz), 7.53-7.62 (4H, m), 8.07 (1H, d, J=3.5 Hz), 8.12 (1H, d J=3.5 Hz), 11.14 (1 H, s); m/z 368.1 [MH⁺]

### b) 4-{[(4-Biphenylyloxy)acetyl]amino}-3-thiophenecarboxylic acid

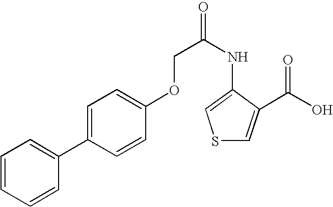

(Method B)

A mixture of methyl 4-{[(4-biphenylyloxy)acetyl]amino}-3-thiophenecarboxylate (51 mg, 0.139 mmol, 1 equiv) and pyridine hydrochloride (0.032 mg, 0.278 mmol, 2 equiv) in anhydrous pyridine (1 ml) were heated together at 110° C. for 18 hrs. The reaction mixture was then allowed to cool to room temperature, acidified with 2M HCl (20 ml) and extracted EtOAc (2×30 ml). The combined organic extracts were dried (MgSO₄) filtered and evaporated. Purification by aminopropyl SPE (5 g) loading the compound and washing with MeOH/CH₂Cl₂ and then eluting with 2M ammonia in MeOH solution. Evaporation of the ammonia solution afforded the title compound as a white solid (40 mg, 81%). ¹H NMR δ_{H} (400 MHz; DMSO-d⁶) 4.81 (2H, s), 7.15 (2H, app d, J=8.5 Hz), 7.32 (1H, t, J=7.5 Hz), 7.44 (2H, t, J=7.5 Hz), 7.60-7.69 (4H, m), 8.00 (1H, d, J=3.5 Hz), 8.34 (1H, d, J=3.5 Hz), 11.22 (1H, s), 13.58 (1H, br s); m/z 354.0 [MH⁺].

### Example 2

### 3-{[(4-Biphenylyloxy)acetyl]amino}-4-methyl-2-thiophenecarboxylic acid

### a) Methyl 3-{[(4-biphenylyloxy)acetyl]amino}-4-methyl-2-thiophenecarboxylate

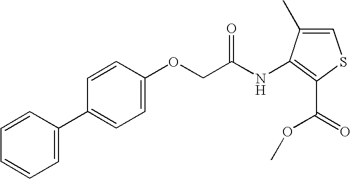

Method A using methyl 3-amino-4-methyl-2-thiophenecarboxylate (0.107 g, 0.626 mmol), and (4-biphenylyloxy)acetic acid (J. Med. Chem 1976, 19, 1079-1088) (0.119 g, 0.521 mmol). Purification by Biotage™ chromatography (Si, 50 g) eluting 19-9:1 cyclohexane:ethylacetate afforded the title compound as a white solid (0.123 g, 62%). ¹H NMR δ_{H} (400 MHz; CDCl₃) 2.26 (3H, s), 3.86 (3H, s), 4.73 (2H, s), 7.11-7.15 (2H, m), 7.18 (1H, br s), 7.31-7.37 (1H, m), 7.44 (2H, app t, J=7.5 Hz), 7.55-7.62 (4H, m), 9.70 (1H, br s); m/z 382.0 [MH⁺].

### b) 3-{[(4-Biphenylyloxy)acetyl]amino}-4-methyl-2-thiophenecarboxylic acid

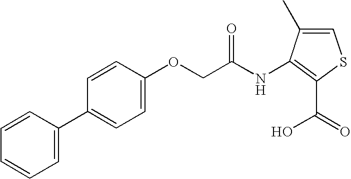

Method B using methyl 3-{[(4-biphenylyloxy)acetyl]amino}-4-methyl-2-thiophenecarboxylate (0.123 g, 0.322 mmol). Purification by aminopropyl SPE (5 g) loading the compound and washing with MeOH/CH₂Cl₂ and then eluting the title compound with 2M ammonia in MeOH solution. Evaporation of the ammonia solutions gave a solid residue that was treated with 1M HCl (10 ml) and then extracted into EtOAc (50 ml). The organic extract was dried (MgSO₄) filtered and evaporated to afford the title compound as a cream solid (55.5 mg, 47%). ¹H NMR δ_{H} (400 MHz; DMSO-d⁶) 2.05 (3H, s), 4.77 (2H, s), 7.12 (2H, app d, J=8.5 Hz), 7.32 (1H, app t, J=7.5 Hz), 7.44 (2H, app t, J=7.5 Hz), 7.51 (1 H, brs), 7.59-7.66 (4H, m), 9.93 (1H, s), 13.10 (1H, brs); m/z 368.1 [MH⁺].

### Example 3

### 2-{[(4-biphenylyloxy)acetyl]amino}-5-methyl-3-thiophenecarboxylic acid

### a) Methyl 2-{[(4-biphenylyloxy)acetyl]amino}-5-methyl-3-thiophenecarboxylate

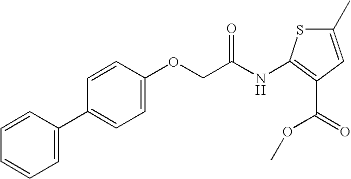

Method A using methyl 2-amino-5-methyl-3-thiophenecarboxylate (0.1 g, 0.585 mmol) and (4-biphenylyloxy)acetic acid (0.111 g, 0.487 mmol). Purification by Biotage™ chromatography (Si, 50 g) eluting 19-9:1 cyclohexane:ethylacetate afforded the title compound as a pale yellow solid (0.142 g, 76%). ¹H NMR δ_{H} (400 MHz;CDCl₃) 2.41 (3H, s), 3.90 (3H, s), 4.77 (2H, s), 6.89 (1 H, br s), 7.11-7.17 (2H, m), 7.34 (1H, app t, J=7.5 Hz), 7.44 (2H, app t, J=7.5 Hz), 7.54-7.62 (4H, m), 11.83 (1H, s); m/z 382.0 [MH⁺].

### b) 2-{[(4-biphenylyloxy)acetyl]amino}-5-methyl-3-thiophenecarboxylic acid

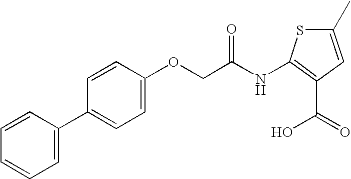

Method B using methyl 2-{[(4-biphenylyloxy)acetyl]amino}-5-methyl-3-thiophenecarboxylate (0.142 g, 0.372 mmol). Purification by aminopropyl SPE (5 g) loading the compound and washing with MeOH/CH₂Cl₂ and then eluting with 5-50% acetic acid in MeOH. Fractions containing product were combined and evaporated to afford the title compound as a beige solid (12 mg, 9%). ¹H NMR δ_{H} (400 MHz; DMSO-d⁶) 2.32 (3H, s), 4.84 (2H, s), 6.81 (1H, br s), 7.14 (2H, app d, J=8.5 Hz), 7.35 (1H, app t, J=7.5 Hz), 7.43 (2H, app t, J=7.5 Hz), 7.62 (4H, m), CO₂H and NH not observed up to δ_{H}=13; m/z 368.1 [MH⁺].

### Example 4

### 4-{[3-(6-phenyl-3-pyridazinyl)propanoyl]amino}-3-thiophene carboxylic acid

### a) 3-(6-phenyl-3-pyridazinyl)propanoic acid

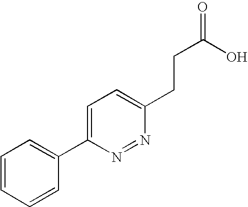

A mixture of 4,7-dioxo-7-phenylhetanoic acid (4.8 g, 20.5 mmol) and hydrazine hydrate (1 ml, 20.6 mmol) was heated in an open flask on a steam bath for 2 hours. The residue was dissolved in boiling dioxan (90 ml) and the crystals which formed on cooling collected. The mother liquors containing the title compound were evaporated and purified by chromatography on silica (100 g) eluting 80:40:1 ethylacetate:cyclohexane:acetic acid. The product obtained was then crystalised from ethyl acetate to afford the title compound as a crystaline solid (0.29 g, 6%). ¹H NMR δ_{H} (400 MHz; DMSO-d⁶) 2.18 (2H, t, J=7.5 Hz), 3.19 (2H, t, J=7.5 Hz), 7.48-7.59 (3H, m), 7.70 (1H, d, J=9 Hz), 8.09-8.18 (3H, m), 12.22 (1H, brs).

### b) Methyl-4-{[3-(6-phenyl-3-pyridazinyl)propanoyl]amino}-3-thiophenecarboxylate

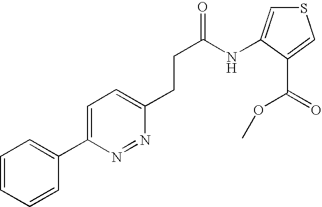

Method B using methyl-4-amino thiophene-3-carboxylate (56.2mg, 0.36 mmol) and 3-(6-phenyl-3-pyridazinyl)propanoic acid (98 mg, 0.43 mmol). Purification by Biotage™ chromatography (Si, 25 g) eluting 1:1 cyclohexane:ethylacetate afforded the title compound as a pale yellow solid (25 mg, 19%). ¹H NMR δ_{H} (400 MHz; CDCl₃) 3.14 (2H, t, J=7 Hz), 3.45 (2H, t, J=7 Hz), 3.89 (3H, s), 7.47-7.56 (4H, m), 7.79 (1H, d, J=8.5 Hz), 8.01 (1H, d, J=3.5 Hz), 8.02 (1H, d, J=3.5 Hz), 8.07 (2H, app d, J=7.5 Hz), 10.11 (1H, brs); m/z 368.1 [MH⁺].

### c) 4-{[3-(6-phenyl-3-pyridazinyl)propanoyl]amino}-3-thiophene carboxylic acid

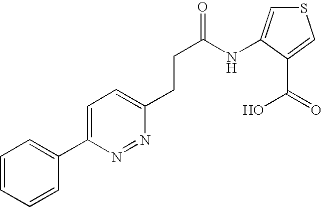

To a solution of methyl 4-{[3-(6-phenyl-3-pyridazinyl)propanoyl]amino}-3-thiophenecarboxylate (25 mg, 0.068 mmol) in MeOH (1 ml) and THF (1 ml) was added 2M NaOH (0.2 ml). The resulting mixture was shaken for 45 minutes and then concentrated in vacuo. The reaction mixture was then acidified 1M HCl (5 ml) and extracted with EtOAc (2×20 ml). The combined organic extracts were washed with brine, dried (MgSO₄) filtered and concentrated in vacuo to afford the title compound as a yellow solid (12 mg, 50%). ¹H NMR δ_{H} (400 MHz; DMSO-d⁶) 3.01 (2H, t, J=7 Hz), 3.31 (2H, t, J=7 Hz), 7.50-7.59 (3H, m), 7.77 (1H, d, J=8.5 Hz), 7.89 (1H, d, J=3.5 Hz), 8.08-8.15 (2H, m), 8.19 (1H, d, J=8.5 Hz), 8.30 (1H, d, J=3.5 Hz), 10.20 (1H, s), CO₂H not observed up to δ_{H}=13; m/z 354.1 [MH⁺].

### Example 5

### 4-{[3-(4-biphenylyl)propanoyl]amino}-3-thiophenecarboxylic acid

### a) Methyl-4-{[3-(4-biphenylyl)propanoyl]amino)-3-thiophenecarboxylate

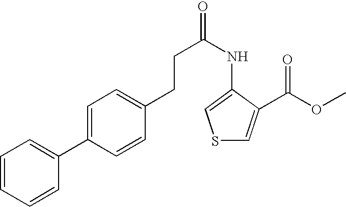

Method A using methyl-4-aminothiophene-3-carboxylate (204 mg, 1.3 mmol) and 3-(4-biphenylyl)propanoic acid (0.267 g, 1.2 mmol). Purification by Biotage™ chromatography (Si, 25 g) eluting 9:1 cyclohexane:ethylacetate afforded the title compound as a pale yellow solid (0.25 g, 57%). ¹H NMR δ_{H} (400 MHz;CDCl₃) 2.78 (2H, t, J=8 Hz), 3.13 (2H, t, J=8 Hz), 3.88 (3H, s), 7.30-7.38 (3H, m), 7.44 (2H, t, J=7.5 Hz), 7.51-7.61 (4H, m), 8.03 (1H, d, J=3.5 Hz), 8.06 (1H, d, J=3.5 Hz), 10.04 (1H, br s); m/z 366.1 [MH⁺].

### b) 4-{[3-(4-biphenylyl)propanoyl]amino}-3-thiophenecarboxylic acid

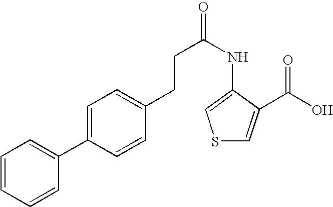

To a solution of methyl 4-{[3-(4-biphenylyl)propanoyl]amino}-3-thiophenecarboxylate (0.245 g, 0.67 mmol) in MeOH (4 ml) and THF (4 ml) was added 2M NaOH (1 ml). The resulting mixture was shaken for 3 hours and then concentrated in vacuo. The residue was acidified 1M HCl (20 ml) and extracted with EtOAc (2×40 ml). The combined organic extracts were washed with brine dried (MgSO₄) filtered and concentrated in vacuo to afford the title compound as a beige solid (0.185 g, 79%). ¹H NMR δ_{H} (400 MHz; δ: DMSO-d⁶) 2.76 (2H, t, J=7.5 Hz), 2.97 (2H, t, J=7.5 Hz) 7.31-7.39 (3H, m), 7.44 (2H, t, J=8 Hz), 7.57 (2H, d, J=8 Hz), 7.63 (2H, d, J=7.5 Hz), 7.91 (1H, d, J=3.5 Hz), 8.29 (1H, d, J=3.5 Hz), 10.14 (1H, s), 13.31 (1H, v brs); m/z 352.1 [MH⁺].

### Example 6

### 2-{[(4-biphenylyloxy)acetyl]amino}-3-thiophenecarboxylic acid

### a) Methyl 2-{[(4-biphenylyloxy)acetyl]amino}-3-thiophenecarboxylate

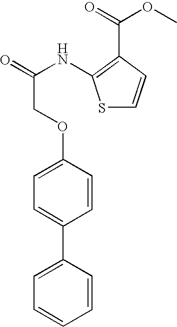

Method A using methyl 2-amino-thiophene-3-carboxylate (0.245 g, 1.56 mmol) and (4-biphenylyloxy)acetic acid (0.296 g, 1.3 mmol). Purification by Biotage™ chromatography (Si, 50 g) eluting 19:1 cyclohexane:ethylacetate afforded the title compound as a white solid (0.256 g, 54%). ¹H NMR δ_{H} (400 MHz; CDCl₃) 3.93 (3H, s), 4.79 (2H, s), 6.81 (1H, d, J=5.5 Hz), 7.12-7.18 (2H, m), 7.25 (1H, d, J=5.5 Hz), 7.34 (1H, m), 7.44 (2H, app t, J=7.5 Hz), 7.54-7.64 (4H, m), 11.96 (1H, br s); m/z 385.1 [MNH₄³⁰ ].

### b) 2-{[(4-biphenylyloxy)acetyl]amino}-3-thiophenecarboxylic acid

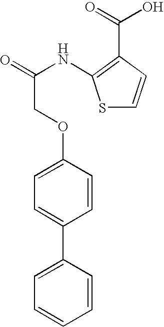

Method B using methyl 2-{[(4-biphenylyloxy)acetyl]amino}-3-thiophenecarboxylate (0.115 g, 0.313 mmol). Purification by aminopropyl SPE (10 g) loading the compound and washing with MeOH/CH₂Cl₂ and then eluting with 2M ammonia in MeOH solution. Evaporation of the ammonia solution gave a solid residue that was treated with 1M HCl (10 ml) and then extracted into EtOAc (50 ml). The organic extract was dried (MgSO₄) filtered and evaporated to afford the title compound as a pale brown solid (47 mg, 43%). ¹H NMR δ_{H} (400 MHz; δ: DMSO-d⁶) 4.96 (2H, s), 7.06 (1H, d, J=5.5 Hz), 7.13-7.22 (3H, m), 7.33 (1H, app t, J=7.5 Hz), 7.41-7.48 (2H, m), 7.60-7.70 (4H, m), 11.94 (1H, br s), 13.42 (1H, v br s); m/z 352.0 [M−H]⁻.

### Example 7

### 3-{[(4-biphenylyloxy)acetyl]amino}-2-thiophenecarboxylic acid

### a) Methyl 3-{[(4-biphenylyloxy)acetyl]amino}-2-thiophenecarboxylate

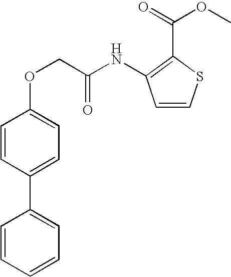

Method D. A mixture of (4-biphenylyloxy)acetyl chloride (F. De Marchi, G. F Tamagnone, F. Dorato, Farmaco, Edizione Scientifica, 1973, 28(7), 511-522)(123 mg, 0.5 mmole, 1 equiv), methyl 3-aminothiophene-2-carboxylic acid, (78.5 mg, 0.5 mmole, 1 equiv) and N,N-diisopropylethylamine (0.109 ml, 81 mg, 0.63 mmole, 1.25 equiv) in anhydrous tetrahydrofuran (5 ml) was stirred overnight at room temperature. The solvent was evaporated and the residue dissolved in dichloromethane (15 ml) and added to a 10 g silica SPE (Varian mega bond elut) and eluted with 1 column volume of dichloromethane. The solvent was evaporated and the residue dissolved in methanol/dichloromethane 1:1 and added to a 10 g Flash SCX-2 SPE (IST Isolute SPE). Elution with methanol/dichloromethane 3:1 gave the title compound as an off white solid (104 mg, 57%); m/z 368.1 [MH⁺], 385.1 [MNH₄⁺].

### b) 3-{[(4-biphenylyloxy)acetyl]amino}-2-thiophenecarboxylic acid

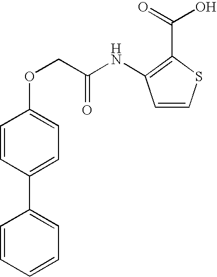

To a solution of methyl 3-{[(4-biphenylyloxy)acetyl]amino}-2-thiophenecarboxylate (50 mg, 0.14 mmol) in MeOH (2 ml) and THF (2 ml) was added lithium hydroxide monohydrate (12.6 mg, 0.299 mmol) as a solution in water (0.5 ml). The resulting mixture was stirred for 20 hours, then further lithium hydroxide monohydrate was added (6.3 mg, 0.15 mmol) as a solution in water (0.5 ml). After a further 4 hours stirring the reaction mixture was concentrated in vacuo and the residue acidified 1M HCl and then extracted EtOAc (2×25 ml). The combined organic extracts were dried (MgSO₄) filtered and concentrated in vacuo. Purification by aminopropyl SPE (5 g) loading the compound and washing with MeOH/CH₂Cl₂ and then eluting the title compound with 5%-20% acetic acid in methanol. Fractions containing pure product were combined and concentrated in vacuo to afford the title compound as a white solid (23 mg, 47%). ¹H NMR δ_{H} (400 MHz; δ: DMSO-d⁶) 4.84 (2H, s), 7.16 (2H, d, J=8.5 Hz), 7.33 (1H, t, J=7.5 Hz), 7.44 (2H, t, J=7.5 Hz), 7.58-7.70 (4H, m), 7.89 (1H, d, J=5 Hz), 8.04 (1H, d, J=5 Hz), 11.30 (1H, br s), 13.65 (1H, vbr s); m/z 352.0 [M−H]⁻

### Example 8

### 2-{[3-(4-biphenylyl)propanoyl]amino}-3-thiophenecarboxylic acid

### a) Methyl 2-{[3-(4-biphenylyl)propanoyl]amino}-3-thiophenecarboxylate

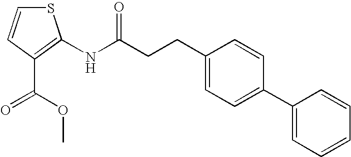

Method A using methyl-2-amino thiophene-3-carboxylate (101 mg, 0.76 mmol) and 3-(4-biphenylyl)propanoic acid (J. Am. Chem. Soc 1972, 94, 4971-4977) (0.144 g, 0.64 mmol). Purification by Biotage™ chromatography (Si, 25 g) eluting 19:1 cyclohexane:ethylacetate afforded the title compound as a white solid (0.125 g, 53%). ¹H NMR δ_{H} (400 MHz;CDCl₃) 2.86 (2H, t, J=8 Hz), 3.14 (2H, t, J=8 Hz), 3.86 (3H, s), 6.74 (1H, d, J=6 Hz), 7.18 (1H, d, J=6 Hz), 7.29-7.37 (3H, m), 7.43 (2H, app t, J=7.5 Hz), 7.51-7.60 (4H, m), 10.97 (1H, br s); m/z 366.0 [MH⁺].

### b) 2-{[3-(4-biphenylyl)propanoyl]amino}-3-thiophenecarboxylic acid

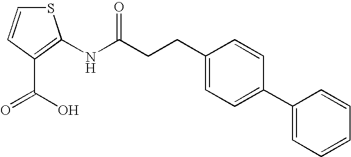

To a solution of methyl 2-{[3-(4-biphenylyl)propanoyl]amino}-3-thiophenecarboxylate (0.123 g, 0.336 mmol) in MeOH (2 ml) and THF (2ml) was added lithium hydroxide monohydrate (31 mg, 0.74 mmol) as a solution in water (1 ml). The resulting mixture was stirred for 20 hours and then concentrated in vacuo. The residue was acidified 1M HCl (20 ml) and then extracted EtOAc (2×30 ml). The combined organic extracts were dried (MgSO₄) filtered and concentrated in vacuo. Purification by aminopropyl SPE (5 g) loading the compound and washing with MeOH/CH₂Cl₂ and then eluting with 5%-20% acetic acid in methanol. Fractions containing pure product were combined and concentrated in vacuo to afford the title compound as a cream solid (23 mg, 20%). ¹H NMR δ_{H} (400 MHz; DMSO-d⁶) 2.87-3.01 (4H, m), 6.93 (1H, d, J=5.5 Hz), 7.12 (1H, d, J=5.5 Hz), 7.29-7.39 (3H, m), 7.43 (2H, t, J=7.5 Hz), 7.57 (2H, d, J=8.5 Hz), 7.63 (2H, d, J=8.5 Hz), 11.26 (1H, br s), CO₂H not observed up to δ_{H}=13; m/z 350.0 [M−H]⁻.

### Example 9

### 4-({[4-(1,2,4-benzotriazin-3-yl)-1-piperazinyl]carbonyl}amino)-3-thiophene carboxylic acid

### a) Methyl 4-({[4-(1,2,4-benzotriazin-3-yl)-1-piperazinyl]carbonyl}amino)-3-thiophenecarboxylate

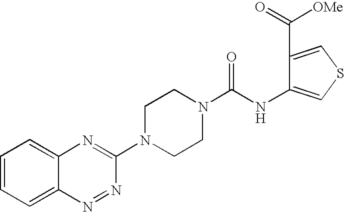

Method E. To a solution of methyl 4-amino-3-thiophenecarboxylate (250 mg, 1.59 mmol, 1 equiv) in anhydrous dioxan (1.5 ml) was added trichloromethylchloroformate (0.44 ml, 3.65 mmol, 2.3 equiv) dropwise under nitrogen whilst a temperature of ˜5° C. was maintained. The reaction mixture was stirred for 1 hour at room temperature before heating at 70° C. for 4 hours. The reaction mixture was cooled to room temperature and dissolved in Et₂O (2 ml) and filtered. This solution was then added to a solution of 3-(1-piperazinyl)-1,2,4-benzotriazine (U.S. Pat. No. 4,091,098 ) (387 mg, 1.8 mmol, 1.13 equiv) in anhydrous dioxan (1.9 ml) under nitrogen. The mixture was stirred for 18 hours at room temperature and then concentrated in vacuo. Purification by Biotage™ chromatography (Si, 50 g) 1:9 EtOAc:cyclohexane, afforded the title compound as an orange solid (169.3 mg, 24%); δ_{H} (400 MHz, DMSO-d⁶) 3.65 (4H, app t, J=7.0 Hz), 3.88 (3H, s), 4.10 (4H, br s), 7.54 (1 H, t, J=7.5 Hz), 7.65 (1H d, J=8.0 Hz), 7.70 (1H, d, J=3.5 Hz), 7.87 (1H, app t, J=7.5 Hz), 8.27 (1H, d, J=8.0 Hz), 8.37 (1H, d, J=3.5 Hz), 9.55 (1H, s); m/z 399.1 [MH⁺].

### b) 4-({[4-(1,2,4-benzotriazin-3-yl)-1-piperazinyl]carbonyl}amino)-3-thiophene carboxylic acid

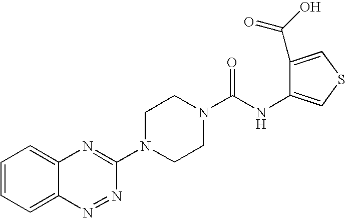

To a solution of methyl 4-({[4-(1,2,4-benzotriazin-3-yl)-1-piperazinyl]carbonyl}amino)-3-thiophenecarboxylate (20 mg, 0.05 mmol, lequiv) in a mixture of THF (0.5 ml) and MeOH (0.5 ml) was added a solution of lithium hydroxide monohydrate (4.1 mg, 0.1 mmol, 2 equiv) in water (0.25 ml). The mixture was stirred at room temperature under nitrogen for 3 hours before being acidified with 2M HCl and extracted with EtOAc (2×25 ml). The organic solution was dried using magnesium sulphate, filtered and concentrated in vacuo to give a solid. Purification by aminopropyl SPE (1 g) loading and washing with methanol, then eluting acetic acid, afforded the title compound as an orange solid (6.6 mg, 34%); δ_{H} (400 MHz, DMSO- d⁶) 3.65 (4H, app t, J=5.0 Hz), 4.09 (4H, br s), 7.53 (1H, app t, J=7.5 Hz), 7.62-7.68 (2H, m), 7.84-7.90 (1H, m), 8.24-9.29 (2H, m), 10.09 (1H, br s), CO₂H not observed up to δ_{H}=13; m/z 385.1 [MH⁺].

### Example 10

### 4-({[4-(1,2,4-benzotriazin-3-yl)-1-piperazinyl]acetyl}amino)-3-thiophene carboxylic acid

### a) Methyl 4-({[4-(1,2,4-benzotriazin-3-yl)-1 -piperazinyl]acetyl}amino)-3-thiophene carboxylate

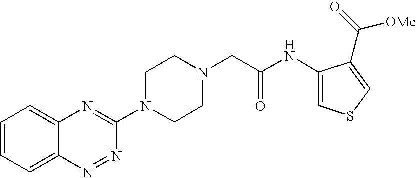

To a solution of methyl 4-[(chloroacetyl)amino]-3-thiophenecarboxylate (European Journal of Medicinal Chemistry 24, 569-572) (150 mg, 0.65 mmol, 1 equiv) in anhydrous DMF (3 ml) under nitrogen was added 3-(1-piperazinyl)-1,2,4-benzotriazine (U.S. Pat. No. 4,091,098)(180 mg, 0.84 mmol, 1.3 equiv) and DIPEA (0.147 ml, 0.84 mmol, 1.3 equiv). The mixture was stirred at 50° C. for 5 hours under nitrogen. After cooling to room temperature, the mixture was partitioned between water and EtOAc. The organic extract was dried (MgSO₄), filtered and concentrated in vacuo. Purification by SPE (Si, 10 g) eluting 9:1 cyclohexane:EtOAc, afforded the title compound as an orange solid (182 mg, 68%); δ_{H} (400 MHz, DMSO) 2.73 (4H, app t, J=5.0 Hz), 3.27 (2H, s), 3.85 (3H, s), 4.13 (4H, br s), 7.50-7.55 (1H, m), 7.63 (1H, d, J=8.5 Hz), 7.83-7.89 (1H, m), 8.05 (1H, d, J=3.5 Hz), 8.25 (1H, dd, J=8.5 and 1 Hz), 8.39 (1H, d, J=3.5 Hz), 11.28 (1H, s); m/z 413.2 [MH⁺].

### b) 4-({[4-(1,2,4-benzotriazin-3-yl)-1 -piperazinyl]acetyl}amino)-3-thiophene carboxylic acid

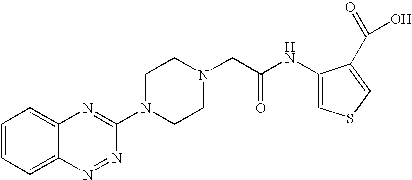

To a solution of methyl 4-({[4-(1,2,4-benzotriazin-3-yl)-1-piperazinyl]acetyl}amino)-3-thiophenecarboxylate (180 mg, 0.44 mmol, 1 equiv) in a mixture of THF (1.5 ml) and MeOH (1.5 ml) was added lithium hydroxide monohydrate (56 mg, 1.32 mmol, 3 equiv) as a solution in water (0.75 ml). The mixture was stirred at room temperature for 36 hours before being acidified using 2M HCl and extracted using EtOAc (2×50 ml). The organic solution was dried using magnesium sulphate, filtered and concentrated in vacuo. Purification by aminopropyl SPE (2 g) loading and washing with methanol, then eluting with acetic acid. Evaporation of the acetic acid afforded the title compound as a yellow/orange solid (48.2 mg, 28%); δ_{H} (400 MHz, DMSO) 2.73 (4H, app t, J=4.5 Hz), 3.25 (2H, s), 4.11 (4H, brs), 7.52 (1H, app t, J=7.5 Hz), 7.63 (1H, d, J=8.5 Hz), 7.86 (1H, appt, J=7.5 Hz), 7.99 (1H, d, J=3.5 Hz), 8.25 (1H, app d, J=8.5 Hz), 8.31 (1H, d, J=3.5 Hz), 11.57 (1H, s), CO₂H not observed up to δ_{H}=13; m/z 399.2 [MH⁺].

### Example 11

### 2-({[1-(1-oxido-1,2,4-benzotriazin-3-yl)-4-piperidinyl]acetyl}amino)-3-thiophenecarboxylic acid

### a) [1-(1-oxido-1,2,4-benzotriazin-3-yl)-4-piperidinyl]acetic acid

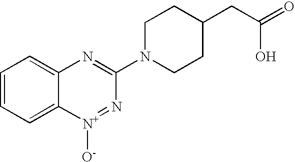

Method F. To a solution of 4-piperidinylacetic acid (444 mg, 3.1 mmol, 1 equiv) in IPA (10 ml) was added 3-chloro-1,2,4-benzotriazine 1-oxide (U.S. Pat. No. 4,091,098) (704 mg, 3.875 mmol, 1.25 equiv) and DIPEA (1.17 ml, 6.2 mmol, 2 equiv). The mixture was stirred at room temperature under nitrogen for 48 hours. Purification by amino propyl SPE (10 g), loading and washing with methanol before eluting with 2M ammonia in methanol. The ammonia extracts were concentrated in vacuo and the residue treated with 2M HCl (75 ml) and then extracted EtOAc (2×150 ml). The combined organic extracts were dried (MgSO₄) filtered and concentrated in vacuo to afford the title compound as an orange solid (894 mg, 100%); δ_{H} (400 MHz, DMSO) 1.10-1.30 (2H, m), 1.78 (2H, app d, J=11 Hz), 1.90-2.05 (1H, m), 2.10-2.20 (2H, m), 3.01 (2H, app t, J=12.5 Hz), 4.56 (2H, app d, J=13 Hz), 7.35 (1H, app t, J=8 Hz), 7.58 (1H, d, 8 Hz), 7.79 (1H, app t, J=7 Hz), 8.13 (1 H, d, J=8.5 Hz), CO₂H not observed up to δ_{H}=13; m/z 289.2 [MH⁺].

### b) Methyl 2-({[1-(1-oxido-1,2,4-benzotriazin-3-yl)-4-piperidinyl]acetyl}amino)-3-thiophenecarboxylate

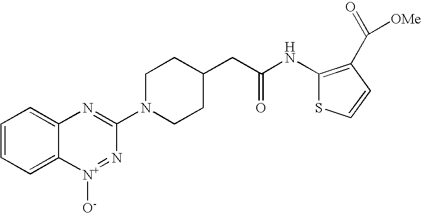

Method A using [1-(1-oxido-1,2,4-benzotriazin-3-yl)-4-piperidinyl]acetic acid (150 mg, 0.52 mmol, 1 equiv) and methyl 2-amino-3-thiophenecarboxylate (107 mg, 0.68 mmol, 1.3 equiv). Purification by Biotage™ chromatography (Si, 50 g) eluting with 1:9 EtOAc:Cyclohexane afforded the title compound as an orange/yellow solid (45 mg, 20%); δ_{H} (400 MHz, DMSO) 1.20-1.35 (2H, m), 1.80 (2H, app d, J=13.0 Hz), 2.08-2.20 (1H, m), 2.57 (2H, app d, J=7.0 Hz), 3.04 (2H, app.t, J=13 Hz), 3.83 (3H, s), 4.57 (2H, app d, J=13.0 Hz), 7.02 (1H, dd, J=6.0 Hz), 7.17 (1H, d, J=6.0 Hz), 7.35 (1H, ddd, J=8.5, 7.5 and 1 Hz), 7.59 (1H, app d, J=8.0 Hz), 7.80 (1H, ddd, J=8.5, 7.5 and 1 Hz), 8.13 (1H, dd, J=8.5 and 0.5 Hz), 10.83 (1 H, s); m/z 428.2 [MH⁺].

### c) 2-({[1-(1-oxido-1,2,4-benzotriazin-3-yl)-4-piperidinyl]acetyl}amino)-3-thiophene carboxylic acid

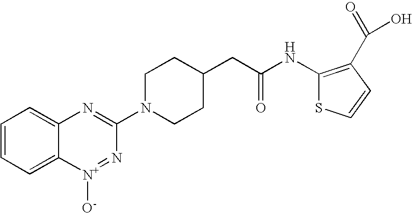

To a solution of methyl 2-({[1-(1-oxido-1,2,4-benzotriazin-3-yl)-4-piperidinyl]acetyl}amino)-3-thiophenecarboxylate (40 mg, 0.094 mmol, 1 equiv) in anhydrous pyridine (1 ml) was added pyridinium hydrochloride (54 mg, 0.48 mmol, 5 equiv). The mixture was stirred at 110° C. for 48 hours. After cooling to room temperature, the mixture was concentrated in vacuo to give a residue which was acidified using 2M HCl and extracted using EtOAc (25 ml). The organic solution was dried (MgSO₄), filtered and concentrated in vacuo. Purification by amino propyl SPE (1 g) loading and washing with methanol and then eluting with 2M ammonia in methanol, afforded the title compound as an orange/yellow solid (15 mg, 40%); δ_{H} (400 MHz, DMSO) 1.20-1.35 (2H, m), 1.79 (2H, app d, J=11.0 Hz), 2.05-2.20 (1H, m), 2.42 (2H, d, J=7.0 Hz), 3.03 (2H, app t t, J=12.5 Hz), 4.58 (2H, app d, J=13.0 Hz), 6.77 (1H, d, J=5.5 Hz), 7.07 (1H, d, J=5.5 Hz), 7.35 (1H, app t, J=7.5 Hz), 7.59 (1H, d, J=8.5 Hz), 7.76-7.83 (1H, m), 8.13 (1H, d, J=8.0 Hz), 12.65 (1H, br s), NH not observed up to δ_{H}=13; m/z 414.2 [MH⁺].

### Example 12

### 2-({[1-(1-oxido-1,2,4-benzotriazin-3-yl)-4-piperidinyl]carbonyl}amino)-3-thiophenecarboxylic acid

### a) 1-(1-oxido-1,2,4-benzotriazin-3-yl)-4-piperidinecarboxylic acid

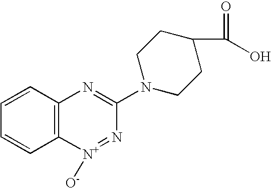

Method F. To a suspension of isonipecotic acid (0.285 g, 2.2 mmol) in isopropanol (20 ml) was added 3-chloro-1,2,4-benzotriazine 1-oxide (U.S. Pat. No. 4,091,098) (0.4 g, 2.2 mmol) and N-N-disopropylethylamine (0.96 ml, 5.5 mmol). The resulting mixture was stirred for 20 hours at room temperature. Isopropanol (20 ml) was then added and the reaction heated at 40° C. for 24 hours, before heating at 50° C. for 3 hours. The reaction mixture was then cooled to room temperature and applied directly to an amino propyl SPE cartridge (50 g). The cartridge was washed methanol, before eluting the compound with 2M ammonia in methanol, which was concentrated in vacuo. The residue was acidified with 1M HCl (40 ml) and extracted EtOAc (2×100 ml). The combined organic extracts were dried (MgSO₄) filtered and evaporated to afford the title compound as an orange solid (0.55 g, 91%) ¹H NMR (400 MHz; δ: DMSO-d⁶) 1.50-1.62 (2H, m), 1.90-1.99 (2H, m), 2.55-2.65 (1H, m), 3.18 (2H, m), 4.45 (2H, m), 7.36 (1H, app t, J=7.5 Hz), 7.59 (1H, d, J=8.5 Hz), 7.80 (1H, app t, J=7.5 Hz), 8.13 (1H, d, J=8.5 Hz), CO₂H not observed up to δ_{H}=13; m/z 275.2 [MH⁺].

### b) Methyl 2-({[1-(1-oxido-1,2,4-benzotriazin-3-yl)-4-piperidinyl]carbonyl}amino)-3-thiophenecarboxylate

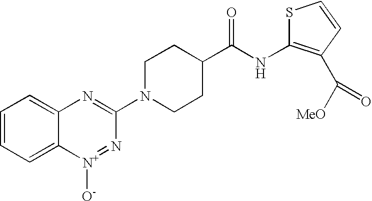

Method A using 1-(1-oxido-1,2,4-benzotriazin-3-yl)-4-piperidinecarboxylic acid (150 mg, 0.55 mmol, 1 equiv) and methyl 2-amino-3-thiophenecarboxylate (113 mg, 0.72 mmol, 1.3 equiv). Purification by Biotage™ chromatography (Si, 12 g) eluting with 1:9 EtOAc:Cyclohexane afforded the title compound as an orange/yellow solid (53 mg, 23%); δ_{H} (400 MHz, DMSO) 1.60-1.78 (2H, m), 2.03 (2H, app d, J=13 Hz), 3.96-3.10 (1H, m), 3.15 (2H, app t, J=13 Hz), 3.85 (3H, s), 4.64 (2H, app d, J=13.0 Hz), 7.03 (1H, d, J=6.0 Hz), 7.18 (1H, d, J=6.0 Hz), 7.34-7.40 (1H, m), 7.62 (1H, d, J=8.0 Hz), 7.78-7.84 (1H, m), 8.12 (1H, app d, J=8.5 Hz), 11.00 (1H, s); m/z414.1 [MH⁺].

### c) 2-({[1-(1-oxido-1,2,4-benzotriazin-3-yl)-4-piperidinyl]carbonyl}amino)-3-thiophene carboxylic acid

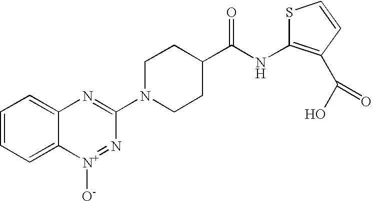

To a solution of methyl 2-({[1-(1-oxido-1,2,4-benzotriazin-3-yl)-4-piperidinyl]carbonyl}amino)-3-thiophenecarboxylate (50 mg, 0.12 mmol, 1 equiv) in anhydrous pyridine (1 ml) was added pyridinium hydrochloride (70 mg, 0.6 mmol, 5 equiv). The mixture was stirred at 110° C. for 48 hours. After cooling to room temperature, the mixture was concentrated in vacuo, acidified using 2M HCl and extracted using EtOAc (25 ml). The organic solution was dried (MgSO₄), filtered and concentrated in vacuo. Purification by amino propyl SPE (1 g) loading and washing with methanol before eluting with 2M ammonia in methanol, afforded the title compound as an orange/yellow solid (35 mg, 73%);67 _{H} (400 MHz, DMSO) 1.58-1.71 (2H, m), 2.02 (2H, dd, J=13.0 and 2.5 Hz), 2.73-2.84 (1H, m), 3.16 (2H, app t, J=12.5 Hz), 4.61 (2H, d, J=13.5 Hz), 6.73 (1H, d, J=5.5 Hz), 7.05 (1H, d, J=5.5 Hz), 7.36 (1H, t, J=7.5 Hz), 7.61 (1H, d, J=8.5 Hz), 7.80 (1H, app t, J=8 Hz), 8.14 (1H, d, J=8.5 Hz), 13.36 (1H, br s) CO₂H not observed up to δ_{H}=13; m/z 400.2 [MH⁺].

### Example 13

### 4-({[1-(1-oxido-1,2,4-benzotriazin-3-yl)-4-piperidinyl]carbonyl}amino)-3-thiophenecarboxylic acid

### a) Methyl 4-({[1-(1-oxido-1,2,4-benzotriazin-3-yl)-4-piperidinyl]carbonyl}amino)-3-thiophenecarboxylate

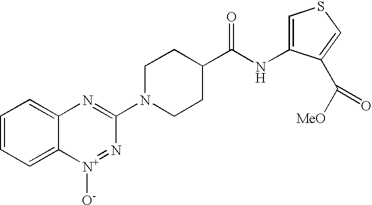

Method B using 1-(1-oxido-1,2,4-benzotriazin-3-yl)-4-piperidinecarboxylic acid (Example 12 a), 150 mg, 0.55 mmol, lequiv) and methyl 4-amino-3-thiophene carboxylate (113 mg, 0.72 mmol, 1.3 equiv). Purification by Biotage™ chromatography (Si, 50 g) eluting with 1:9 EtOAc:Cyclohexane afforded the title compound as an orange/yellow solid (127 mg, 56%); δ_{H} (400 MHz, DMSO) 1.65 (2H, app qd, J=12.0 and 3.5 Hz), 2.00 (2H, dd, J=13.0 and 2.5 Hz), 2.75-2.85 (1H, m), 3.14 (2H, app.t, J=13.5 Hz), 3.86 (3H, s), 4.64 (2H, app d, J=13.5 Hz), 7.37 (1H, app t, J=8.5 Hz), 7.61 (1H, d, J=8.5 Hz), 7.81 (1H, app t, J=8.5 Hz), 7.92 (1H, d, J=3.5 Hz), 8.14 (1H, app d, J=8.5 Hz), 8.37 (1H, d, J=3.5 Hz), 10.04 (1 H, s); m/z 414.1 [MH⁺].

### b) 4-({[1-(1-oxido-1,2,4-benzotriazin-3-yl)-4-piperidinyl]carbonyl}amino)-3-thiophene carboxylic acid

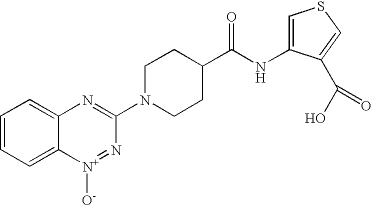

To a solution of methyl 4-({[1-(1-oxido-1,2,4-benzotriazin-3-yl)-4-piperidinyl]carbonyl}amino)-3-thiophenecarboxylate (120 mg, 0.29 mmol, 1 equiv) in a mixture of THF (0.5 ml) and MeOH (0.5 ml) was added lithium hydroxide monohydrate (36 mg, 0.87 mmol, 3 equiv) as a solution in water (0.25 ml). The mixture was stirred at room temperature for 4 hours before being acidified using 2M HCl and extracted using EtOAc (2×25 ml). The organic solution was dried (MgSO₄), filtered and concentrated in vacuo to afford the title compound as an orange solid (77 mg, 67%); δ_{H} (400 MHz, DMSO) 1.60-1.71 (2H, m), 2.95-2.03 (2H, m), 2.70-2.85 (1H, m), 3.16 (2H, app t, J=14.5 Hz), 4.62 (2H, app d, J=13.0 Hz), 7.37 (1H, app t, J=8.5 Hz), 7.61 (1H, d, J=8.5 Hz), 7.81 (1H, app t, J=8.5 Hz), 7.91 (1H, d, J=3.5 Hz), 8.14 (1H, app d, J=8.5 Hz), 8.31 (1H, d, J=3.5 Hz), 10.29 (1H, s), CO₂H not observed; m/z 400.2 [MH⁺].

### Example 14

### 4-({[1-(1-oxido-1,2,4-benzotriazin-3-yl)-4-piperidinyl]acetyl}amino)-3-thiophenecarboxylic acid

### a) Methyl 4-({[1-(1-oxido-1,2,4-benzotriazin-3-yl)-4-piperidinyl]acetyl}amino)-3-thiophenecarboxylate

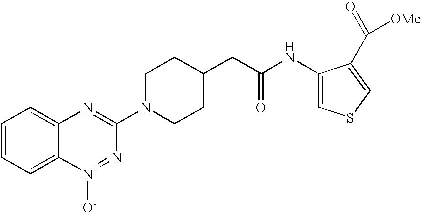

Method A using [1-(1-oxido-1,2,4-benzotriazin-3-yl)-4-piperidinyl]acetic acid (150 mg, 0.52 mmol, 1 equiv) and methyl 4-amino-3-thiophenecarboxylate (107 mg, 0.68 mmol, 1.3 quiv). Purification by Biotage™ chromatography (Si, 50 g) eluting with 1:9 EtOAc:Cyclohexane afforded the title compound as an orange/yellow solid (200 mg, 90%); δ_{H} (400 MHz, DMSO) 1.22-1.34 (2H, m), 1.80 (2H, app d, J=13.0 Hz), 2.05-2.12 (1H, m), 2.38 (2H, d, J=7.0 Hz), 3.04 (2H, app.t, J=13.0 Hz), 3.84 (3H, s), 4.57 (2H, d, 13.0 Hz), 7.35 (1H, app.t J=8.0 Hz), 7.59 (1H, d, J=8.5 Hz), 7.79 (1H, app.t, J=8.0 Hz), 7.93 (1H, d, J=3.5 Hz), 8.13 (1H, d, J=8.5 Hz), 8.36 (1H, d, J=3.5 Hz), 9.87 (1H, s); m/z 428.2 [MH⁺].

### b) 4-({[1-(1-oxido-1,2,4-benzotriazin-3-yl)-4-piperidinyl]acetyl}amino)-3-thiophene carboxylic acid

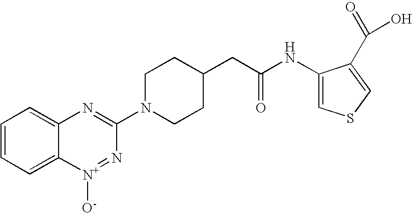

To a solution of methyl 4-({[1-(1-oxido-1,2,4-benzotriazin-3-yl)-4-piperidinyl]acetyl}amino)-3-thiophenecarboxylate (190 mg, 0.44 mmol, 1 equiv) in a mixture of THF (0.5 ml) and MeOH (0.5 ml) was added lithium hydroxide monohydrate (55 mg, 1.33 mmol, 3 equiv) as a solution in water (0.25 ml). The mixture was stirred at room temperature for 4 hours before being acidified using 2M HCl and extracted using EtOAc (2×25 ml). The organic solution was dried (MgSO₄), filtered and concentrated in vacuo to afford the title compound as an orange solid (152 mg, 84%); δ_{H} (400 MHz, DMSO) 1.21-1.33 (2H, m), 1.80 (2H, app d, J=13 Hz), 2.05-2.17 (1H, m), 2.37 (2H, d, J=7.0 Hz), 3.03 (2H, t, J=11.0 Hz), 4.58 (2H, d, J=13 Hz), 7.35 (1H, app.t, J=8.5 Hz), 7.59 (1H, d, J=8.5 Hz), 7.79 (1H, app.t, J=8 Hz), 7.91 (1H, d, J=3.5 Hz), 8.13 (1H, dd, J=9.0 and 1.0 Hz), 8.30 (1H, d, J=3.5 Hz), 10.12 (1H, s), 13.40 (1H, v.brs); m/z 414.1 [MH⁺].

### Example 15

### 4-({[1-(1,2,4-benzotriazin-3-yl)-4-piperidinyl]acetyl}amino)-3-thiophene carboxylic acid

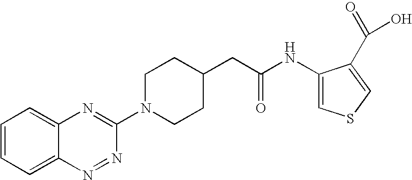

Method F final reduction step. A solution of 4-({[1-(1-oxido-1,2,4-benzotriazin-3-yl)-4-piperidinyl]acetyl}amino)-3-thiophenecarboxylic acid (Example 14 b), 50 mg, 0.12 mmol) in ethanol (5 ml) was hydrogenated using 10% palladium on carbon (50% H₂O, degussa type, 10 mg). After 3 hours the mixture was filtered through Celite and the organic solution concentrated in vacuo. Purification by amino propyl SPE (1 g) loading and washing with methanol before eluting with 2M ammonia in methanol. Evaporation of the ammonia solution afforded a solid which was partitioned between 2M HCl (25 ml) and EtOAc (25 ml). The organic solution was dried (MgSO₄), filtered and concentrated in vacuo to afford the title compound as a yellow solid (16 mg, 34%); δ_{H} (400 MHz, DMSO) 1.23-1.37 (2H, m), 1.85 (2H, d, J=12.0 Hz), 2.17 (1H, m), 2.38 (2H, d, J=7.0 Hz), 3.10-3.20 (2H, m), 4.91 (2H, d, J=11.0 Hz), 7.48 (1H, app.t, J=7.5 Hz), 7.59 (1H, d, J=8.5 Hz), 7.78-7.85 (1H, m), 7.92 (1H, d, J=3.5 Ha), 8.21 (1H, d, J=8 Hz), 8.27 (1H, d, J=3.5 Hz), 10.22 (1H, s), 13.19 (1H, v br s); m/z 398.1 [MH⁺].

### Example 16

### 4-{[(4-biphenylyloxy)acetyl]amino}-3-furancarboxylic acid

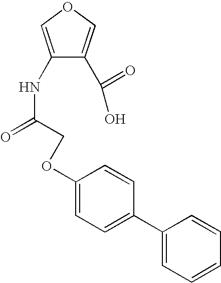

(Method C)

A mixture of 2-(4-biphenylyloxy)acetamide (J. Med. Chem. 1983, 26, 700-714) (0.055 g, 0.242 mmol, 1 equiv), 4-iodo-3-furancarboxylic acid (J. Org. Chem. 1997, 62, 8750-8759) (0.075 g, 0.315 mmol, 1.3 quiv), potassium iodide (0.0604 g, 0.364 mmol, 1.5 equiv), copper powder (0.0285 g, 0.448 mmol, 1.85 equiv) and potassium carbonate (0.0838 g, 0.606 mmol, 2.5 equiv) in DMF (0.75 ml) were heated at 120° C. for 20 hours. The reaction mixture was then allowed to cool to room temperature, treated with 1M HCl (20 ml) and extracted with EtOAc (2×50 ml). The combined organic extracts were washed with brine, dried (MgSO₄), filtered and concentrated in vacuo. Purification by aminopropyl SPE (5 g) loading the compound and washing with MeOH/CH₂Cl₂ and then eluting with 5% acetic acid in methanol. Fractions containing pure product were combined and concentrated in vacuo to afford the title compound as a white solid (45 mg, 55%). ¹H NMR δ_{H} (400 MHz; DMSO-d⁶) 4.84 (2H, s), 7.13 (2H, app d, J=8.5 Hz), 7.32 (1H, app t, J=7.5 Hz), 7.44 (2H, app t, J=7.5 Hz), 7.64 (4H, app t, J=8.5 Hz), 8.24 (1H, brs), 8.29 (1H, br s), 10.08 (1H, br s), 13.54 (1H, br s); m/z 338.2 [MH⁺].

### Example 17

### 3-{[(4-biphenylyloxy)acetyl]amino}-2-furancarboxylic acid

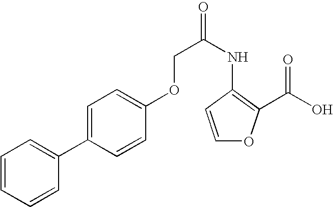

Method C using 3-bromo-2-furancarboxylic acid (Heterocycles, 1993, 36, 1867-1882) (95 mg, 0.5 mmol). Purification by autoprep on a supercosil ABZ (10 cm×21.2 mm) column eluting 35-80%-0.05% formic acid acetonitrile: 0.1% formic acid water at 8 ml/min. Fractions were concentrated in vacuo to afford the title compound as a yellow solid (3.5 mg, 3%). ¹H NMR δ_{H} (400 MHz; δ: DMSO-d⁶) 4.83 (2H, s), 7.13 (2H, d, J=8.5 Hz), 7.28-7.36 (2H, m), 7.44 (2H, t, J=7.5 Hz), 7.64 (4H, app t, J=9 Hz), 7.82 (1H, v br s), 10.24 (1H, v br s), 13.71 (1H, br s); m/z 336.1 [M−H]⁻.

All publications, including but not limited to patents and patent applications, cited in this specification are herein incorporated by reference as if each individual publication were specifically and individually indicated to be incorporated by reference herein as though fully set forth.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

1. A compound selected from:
a compound of Formula (I) 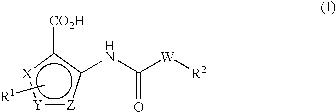
and a salt, or hydrate thereof, wherein:
R¹ represents hydrogen or C₁-C₃alkyl;
R² represents a 5, 6 or 10-member aryl, heteroaryl, heterocyclic or alicyclic ring system;
X, Y and Z independently represent S, O or CH, with the proviso that all three of X, Y and Z cannot represent CH;
W represents —(CH₂)_{q}—; —CH═CH—; —(CH₂)ₚNHC(O)—; —(CH₂)ₚNHC(O)NH—; —(CH₂)ₚNHC(O)O—; —(CH₂)ₚSO₂NR³—; —(CH₂)ₚNR³SO₂—; —(CH₂)ₙO—; —C(R⁴R⁵)O— or -A-B-C-;
A and C, which may independently be present or absent, where present independently represent —(CH2)_{q}—; —CH═CH—; —(CH₂)ₚNHC(O)—; —(CH₂)ₚNHC(O)O—; —(CH₂)ₚNHC(O)NH—; —(CH₂)ₚSO₂NR³—; —(CH₂)ₚNR³SO₂—; —(CH₂)ₚC(O)—; —(CH₂)ₚNH—; —(CH₂)ₚO—; —(CH₂)ₚS— or —(CH₂)ₚO—CH₂—;
B represents a 5 or 6-member aryl, heteroaryl, heterocyclic or alicyclic ring;
n represents an integer selected from 2, 3 and 4;
p represents an integer selected from 0, 1 and 2;
q represents an integer selected from 1, 2, 3 and 4;
R³ represents hydrogen or methyl; and
R⁴ and R ⁵, which may be the same or different, independently represent C₁-C₃alkyl.

2. A compound according to claim 1 wherein R¹ represents hydrogen or methyl.

3. A compound according to claim 1 wherein two of X, Y and Z represent CH.

4. A compound according to claim 1 in which X, Y and Z, together with the carbon atoms to which they are attached, form a heteroaryl ring selected from thiophenyl and furanyl.

5. A compound according to claim 1 wherein R² is selected from benzotriazinyl, cyclohexyl, phenyl, pyridinyl, pyrimidinyl, pyridazinyl and isoxazolyl.

6. A compound according to wherein R² is substituted phenyl.

7. A compound according to claim 6 wherein R² is phenyl substituted with one or two substituents selected from halogen C₁₋₃alkyl, C₁₋₃haloalkyl C₁₋₃alkoxy and C₁₋₃haloakloxy.

8. A compound according to claim 1 wherein R² is selected from the group consisting of:

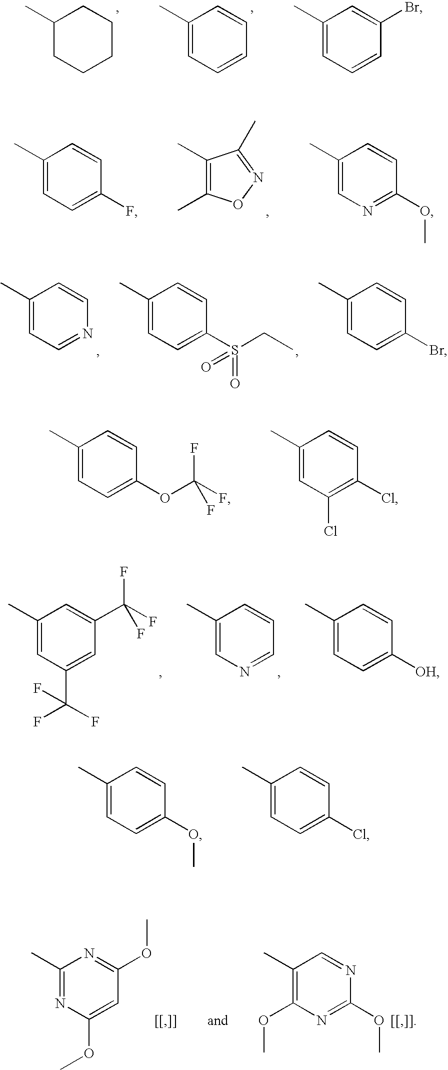

9. A compound according to claim 1 wherein R² is selected from the group consisting of:

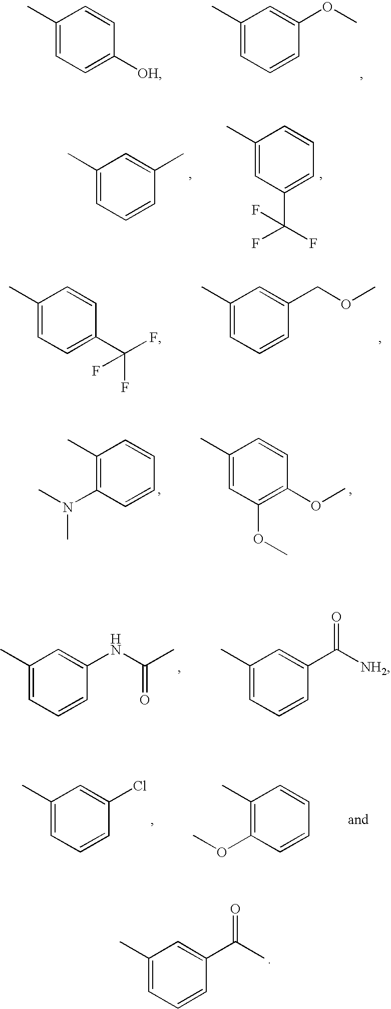

10. A compound according to claim 1 wherein W represents -A-B-C-, —(CH₂)_{q}—, —(CH₂)ₙO— or —(CH₂)ₚNHC(O)—.

11. A compound according to claim 10 wherein W represents -A-B-C-, —O—, —CH₂— or —CH₂O—.

12. A compound according to claim 11 wherein W represents -A-B-C- and A is absent or represents —(CH₂)ₚSO₂NR³—, —(CH₂)ₚNHC(O)— or —(CH₂)ₚNHC(O)NH—.

13. A compound according to claim 11 wherein W represents -A-B-C-, A represents —CH₂— and C represents —(CH₂)ₚSO₂NR³—.

14. A compound according to claim 1 wherein A represents —O— or —CH₂O— and C is absent.

15. A compound according to claim 1 wherein B represents a 5 or 6 member aryl or heteroaryl ring.

16. A compound according to claim 15 wherein B represents phenyl.

17. A compound according to claim 15 wherein B represents a 5 member heteroaryl ring.

18. A compound according to claim 15 wherein B represents a 5 member heterocyclic ring.

19. A compound according to claim 15 wherein B represents benzotriazinyl.

20-24. (canceled)

25. A method for the treatment of a human or animal subject having a condition characterised by under-activation of the HM74A receptor or in which activation of the receptor will be beneficial, which method comprises administering to said human or animal subject an effective amount of the compound according to claim 1.

26. A method according to claim 25 wherein the condition is a disorder of lipid metabolism including dislipidaemia or hyperlipoproteinaemia or an inflammatory disease or condition.

27. A pharmaceutical formulation comprising the compound according to claim 1 in admixture with one or more physiologically acceptable diluents, excipients or carriers.

28. A combination for administration together or separately, sequentially or simultaneously in separate or combined pharmaceutical formulations, said combination comprising the compound according to claim 1 together with another therapeutically active agent.

29. A pharmaceutical formulation comprising the compound according to claim 1 plus a further active ingredient selected from the group consisting of a statin, a fibrate, a bile-acid binding resin and nicotinic acid and one or more physiologically acceptable diluents, excipients or carriers.


SEQUENCE LISTING (1 sequence)

SEQ ID NO: 1 - DNA, 1295 bp, Homo sapien
cgccactttgctggagcattcactaggcgaggcgctccatcggactcactagccgcactcatgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactatttttccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaaataaccatgccaagaagggacattgtcaccaagaaccagcatctctggagaaacagttgggctgttgcatcgagtaatgtcactggactcggcctaaggtttcctggaacttccagattcagagaatctgatttagggaaact